(19) United States
(12) Patent Application Publication
(10) Pub. No.: US 2008/0021462 A1
(43) Pub. Date: Jan. 24, 2008

(54) Spinal stabilization implants

(75) Inventor: Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: WARSAW ORTHOPEDIC INC., Warsaw, IN (US)

(21) Appl. No.: 11/491,765

(22) Filed: Jul. 24, 2006

(51) Int. Cl. A61F 2/30

(52) U.S. Cl. 606/61

(57) ABSTRACT

In an exemplary embodiment, an implantable device is provided which includes a first component configured to fixably attach to a first vertebra to secure the first vertebra in a position relative to a second vertebra. The component also includes a polymeric material including a rigid-rod polymer.

### FIELD OF THE DISCLOSURE

This disclosure, in general, relates to implantable devices and particularly to long-term implantable devices for implantation in and around the spine.

### BACKGROUND

In human anatomy, the spine is a generally flexible column that can withstand tensile and compressive loads. The spine also allows bending motion and provides a place of attachment for keels, muscles, and ligaments. Generally, the spine is divided into four sections: the cervical spine, the thoracic or dorsal spine, the lumbar spine, and the pelvic spine. The pelvic spine generally includes the sacrum and the coccyx. The sections of the spine are made up of individual bones called vertebrae. Three joints reside between each set of two vertebrae: a larger intervertebral disc between the two vertebral bodies and two zygapophysial joints located posteriolaterally relative to the vertebral bodies and between opposing articular processes.

The intervertebral discs generally function as shock absorbers and as joints. Further, the intervertebral discs can absorb the compressive and tensile loads to which the spinal column can be subjected. At the same time, the intervertebral discs can allow adjacent vertebral bodies to move relative to each other, particularly during bending or flexure of the spine. Thus, the intervertebral discs are under constant muscular and gravitational stress and generally, the intervertebral discs are the first parts of the lumbar spine to show signs of deterioration.

The zygapophysial joints permit movement in the vertical direction, while limiting rotational motion of two adjoining vertebrae. In addition, capsular ligaments surround the zygapophysial joints, discouraging excess extension and torsion. In addition to intervertebral disc degradation, zygapophysial joint degeneration is also common because the zygapophysial joints are frequently in motion with the spine. In fact, zygapophysial joint degeneration and disc degeneration frequently occur together. Generally, although one can be the primary problem while the other is a secondary problem resulting from the altered mechanics of the spine, by the time surgical options are considered, both zygapophysial joint degeneration and disc degeneration typically have occurred.

Deterioration of the spine in general can be manifested in many different forms, including, spinal stenosis, degenerative spondylolisthesis, degenerative scoliosis, or a herniated disc, or sometimes a combination of these problems. Accordingly the industry continues to seek new ways to prevent and improve the condition of the spine in patients. Particularly, the medical industry seeks improved devices and procedures to combat the various maladies associated with the spine.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of a lateral view of a portion of a vertebral column.

FIG. 2 includes an illustration of a lateral view of a pair of adjacent vertebrae.

FIG. 3 includes an illustration of a top plan view of a vertebra.

FIG. 4 includes an illustration of a top view of an intervertebral disc.

FIG. 5 includes an illustration of a cross-sectional view of two adjacent vertebrae.

FIG. 6 includes an illustration of an exemplary rod according to one embodiment.

FIG. 7 includes an illustration of an exemplary threaded rod according to one embodiment.

FIG. 8 includes an illustration of an exemplary threaded rod with scoring.

FIG. 9 includes an illustration of an exemplary threaded rod with a movable portion.

FIG. 10 includes an illustration of an exemplary bolt.

FIG. 11 includes an illustration of an exemplary screw.

FIG. 12 includes an illustration of an exemplary coupling device.

FIG. 13, FIG. 14, and FIG. 15 include illustrations of an exemplary screw and rod device.

FIG. 16 includes an illustration of an exemplary polyaxial screw.

FIG. 17 includes an illustration of an exemplary pedicle screw.

FIG. 18 includes an illustration of an exemplary screw and rod device.

FIGS. 19 and 21 include illustrations of exemplary mesh devices.

FIG. 20 includes an illustration of an exemplary strand device.

FIG. 22 and FIG. 23 include illustrations of exemplary plate devices.

FIG. 24 includes an illustration of an exemplary stabilization system.

FIG. 25 and FIG. 26 include illustrations of an exemplary interspinous process braces.

FIG. 27, FIG. 28, FIG. 29, FIG. 30, and FIG. 31 include illustrations of exemplary fusion devices.

FIG. 32, FIG. 33, FIG. 34, FIG. 35, FIG. 36, FIG. 37, and FIG. 38 include illustrations of exemplary disc prosthetic devices.

FIG. 39 includes an illustration of an exemplary device kit.

FIG. 40, FIG. 41, and FIG. 42 include illustrations of an exemplary embodiment of a lumbar brace.

FIG. 43, FIG. 44, FIG. 45, FIG. 46, and FIG. 47 include illustrations of exemplary embodiments of a porous fusion device.

FIG. 48 includes an illustration of an exemplary rod device.

The use of the same reference symbols in different drawings indicates similar or identical items.

### DESCRIPTION OF THE DRAWINGS

In a particular embodiment, an implantable device is configured to secure at least one vertebra in a fixed position relative to a second vertebra. The implantable device can include a component that is formed of a polymeric material including a rigid-rod polymer. In particular examples, the component can include a screw, a rod, a fusion device, a plate, or a prosthetic disc.

In an exemplary embodiment, an implantable device is provided which includes a first component configured to fixably attach to a first vertebra to secure the first vertebra in a position relative to a second vertebra. The component also includes a polymeric material including a rigid-rod polymer.

In another exemplary embodiment, an implantable device includes a component configured to fixably attach to a first vertebra. The component includes a polymer material having a specific gravity of not greater than about 1.40 and an ultimate tensile strength at room temperature (23° C.) of at least about 100 MPa.

In a further exemplary embodiment, an implantable device includes a component configured for location in proximity to a first vertebra. The component is formed of a polymeric material comprising a rigid-rod polymer matrix.

In an additional embodiment, an implantable device includes a first component comprising a rigid rod polymer material and having a first major and opposing engagement surface and a second major and opposing engagement surface. The first and second major and opposing engagement surfaces are configured to fixably engage an upper vertebra and a lower vertebra.

In a further exemplary embodiment, a spinal implant device is provided that includes a rod component, and a screw component configured to fixably attach to a vertebra and to the rod component. At least one of the rod component or the screw component comprises a rigid rod polymer material.

In a further exemplary embodiment, a medical kit includes a component of an implantable device and a tool. The component is configured to fixably attach to a first vertebra to secure the first vertebra in a position relative to a second vertebra. The component includes a polymeric material including a rigid-rod polymer. The tool is configured for use in association with fixably attaching the component to the first vertebra.

In an additional exemplary embodiment, a method of implanting a medical device includes preparing the medical device for implantation. The implantable device includes a component configured to fixably attach to a first vertebra to secure the first vertebra in a position relative to a second vertebra. The component includes a polymeric material including a rigid-rod polymer. The method also includes fixably attaching the component to the first vertebra.

### Description of Relevant Anatomy

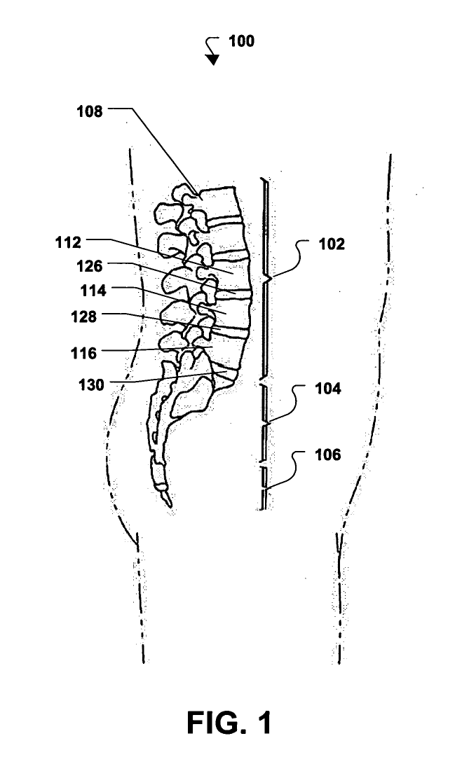

Referring initially to FIG. 1, a portion of a vertebral column, designated 100, is shown. As depicted, the vertebral column 100 includes a lumbar region 102, a sacral region 104, and a coccygeal region 106. The vertebral column 100 also includes a cervical region and a thoracic region. For clarity and ease of discussion, the cervical region and the thoracic region are not illustrated.

As illustrated in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, or damaged or if one of the zygapophysial joints is diseased, degenerated or damaged, that disc or joint can be at least partially treated with an implanted device according to one or more of the embodiments described herein. In a particular embodiment, a fusion device or a fixation device can be inserted into the intervertebral lumbar disc 122, 124, 126, 128, 130 or a zygapophysial joint.

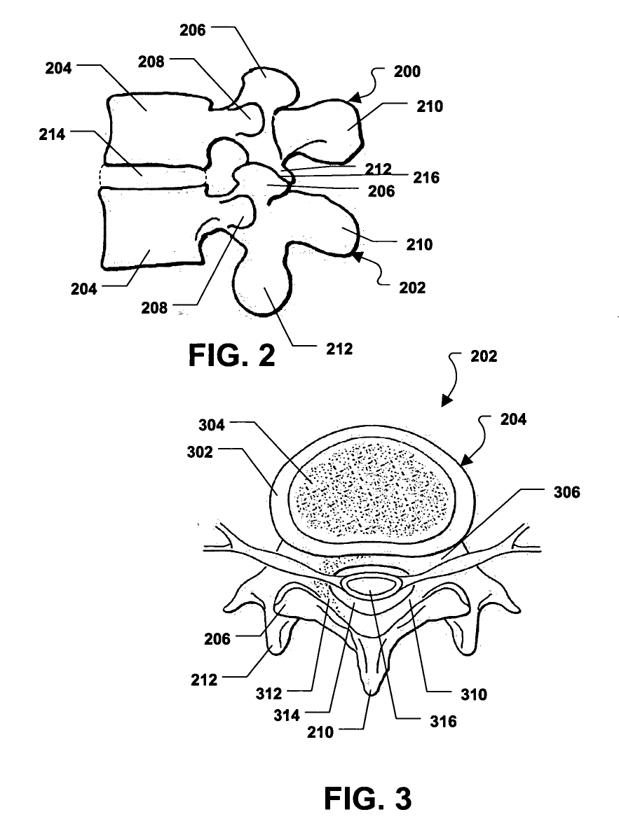

FIG. 2 depicts a detailed lateral view of two adjacent vertebrae, e.g., two of the lumbar vertebrae 108, 110, 112, 114, 116 illustrated in FIG. 1. FIG. 2 illustrates a superior vertebra 200 and an inferior vertebra 202. As illustrated, each vertebra 200, 202 includes a vertebral body 204, a superior articular process 206, a transverse process 208, a spinous process 210 and an inferior articular process 212. FIG. 2 further depicts an intervertebral disc 214 between the superior vertebra 200 and the inferior vertebra 202. A zygapophysial joint 216 is located between the inferior articular process 212 of the superior vertebra 200 and the superior articular process 206 of the inferior vertebra 202. As described in greater detail below, an implantable device according to one or more of the embodiments described herein can be installed within or in proximity to the intervertebral disc 214 between the superior vertebra 200 and the inferior vertebra 202 or within or in proximity to the zygapophysial joint 216.

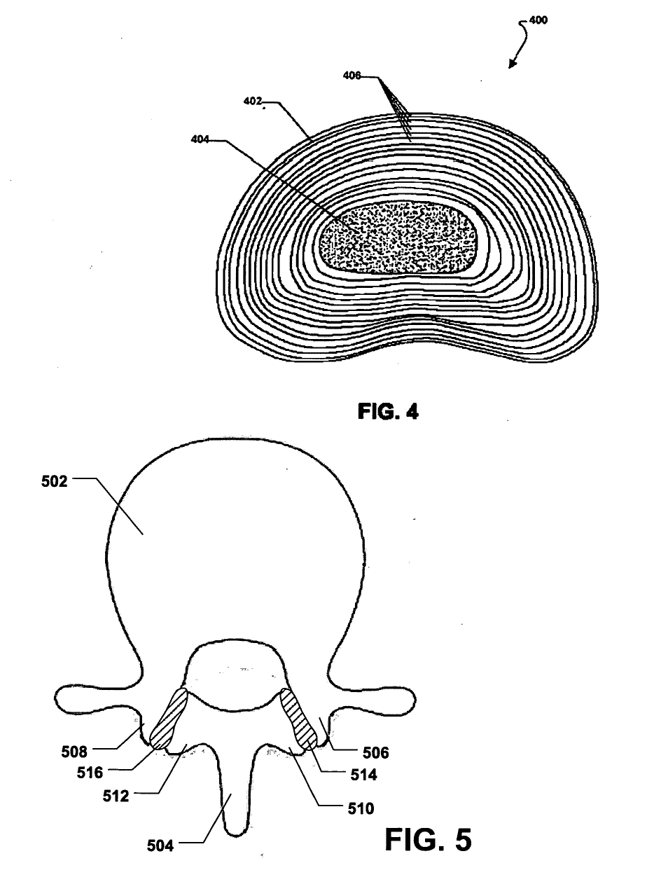

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is generally softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

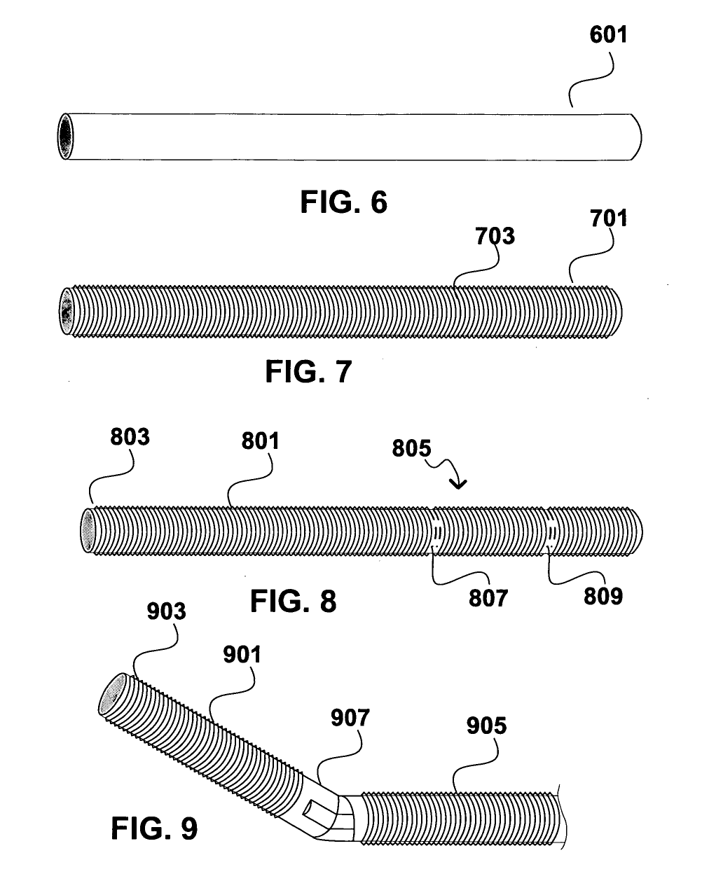

Referring now to FIG. 4, an intervertebral disc is shown and is generally designated 400. The intervertebral disc 400 is made up of two components: an annulus fibrosis 402 and a nucleus pulposus 404. The annulus fibrosis 402 is the outer portion of the intervertebral disc 400, and the annulus fibrosis 402 includes a plurality of lamellae 406. The lamellae 406 are layers of collagen and proteins. Each lamella 406 typically includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is an inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 can allow the nucleus pulposus 404 to be squeezed through the annulus fibers either partially, causing the disc to bulge, or completely, allowing the disc material to escape the intervertebral disc 400. The bulging disc or nucleus material can compress the nerves or spinal cord, causing pain. Accordingly, the nucleus pulposus 404 can be treated or replaced with an implantable device to improve the condition of the intervertebral disc 400.

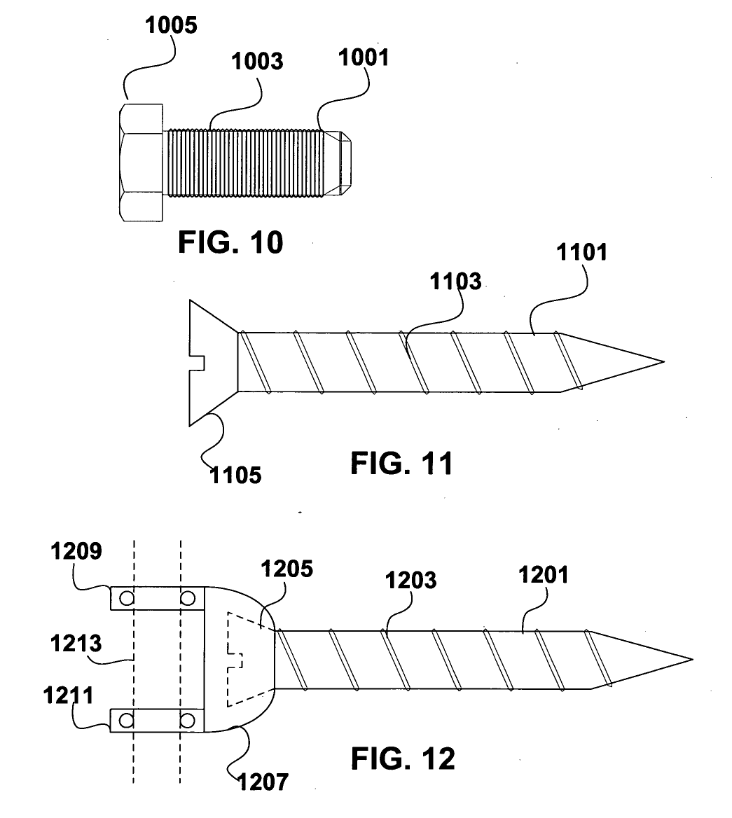

FIG. 5 includes a cross-sectional view of the spine illustrating a portion of a superior vertebra 504 and a portion of an inferior vertebra 502. The inferior vertebra 502 includes superior articular processes 506 and 508 and the superior vertebra 504 includes inferior articular processes 510 and 512. Between the superior articular process 506 and the inferior articular process 510 is a zygapophysial joint 514 and between the superior articular process 508 and the inferior articular process 512 is a zygapophysial joint 516.

When damaged or degraded, the zygapophysial joints 514 and 516 can be treated. For example, an implantable device can be inserted into or in proximity to the zygapophysial joints 514 and 516. In particular, such an implantable device can be configured to fuse or fix the inferior articular process (506 or 508) to the superior articular process (510 or 512).

### Description of Materials for Use in Implantable Devices

In general, components of implantable devices are formed of biocompatible materials. For example, components can be formed of a metallic material, a ceramic material, a polymeric material, or any combination thereof. An exemplary metallic material includes titanium, titanium alloy, tantalum, tantalum alloy, zirconium, zirconium alloy, stainless steel, cobalt, cobalt containing alloy, chromium containing alloy, indium tin oxide, silicon, magnesium containing alloy, aluminum, aluminum containing alloy, or any combination thereof.

An exemplary ceramic material includes an oxide, a carbide, a nitride, or any combination thereof. More particularly, a ceramic can include an oxide, for example, aluminum oxide, zirconium oxide, or any combination thereof. An exemplary carbide includes titanium carbide. A ceramics can also include a carbon containing compound, including graphite, carbon fiber, pyrolytic carbon, diamond, or any combination thereof.

The polymer materials of components of implantable devices are generally biocompatible. An exemplary polymeric material can include a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. Particular polymers are also resorbable in vivo and a resorbable polymer can be gradually moved from the implantable device, either through degradation or solvent effects produced in vivo.

An exemplary polyolefin material can include polypropylene, polyethylene, halogenated polyolefin, flouropolyolefm, polybutadiene, or any combination thereof. An exemplary polyaryletherketone (PAEK) material can include polyetherketone (PEK), polyetheretherketone (PEEK), polyetherketoneketone (PEKK), polyetherketoneetherketoneketone (PEKEKK), or any combination thereof. An exemplary silicone can include dialkyl silicones, fluorosilicones, or any combination thereof. An exemplary hydrogel can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof.

In a particular embodiment, a component of the device includes a rigid-rod polymer. In particular, the rigid-rod polymer can be a phenylene-based polymer, such as a homopolymer or a copolymer in which phenylene forms a portion of the polymeric chain in contrast to forming a functional group extending from the polymeric chain. Depending on the nature of copolymer monomers and functional groups, a rigid-rod polymer can form a crystalline phase that can provide strength or can provide conductivity.

Particular rigid-rod polymers can include copolymers that, in addition, to a phenylene group, include a benzoyl, an azole, a thiazole, an oxazol, a terephthalate group, or any combination thereof in the polymer chain. In a particular example, the rigid-rod polymer can include poly(phenylene benzobisthiazole) (PPBT), such as poly(p-phenylene benzobisthiazole). In another example, the rigid-rod polymer can include poly(phenylene benzobisoxazole) (PBO), such as poly(p-phenylene benzobisoxazole). In a further example, the rigid-rod polymer can include poly(phenylene benzimidazole) (PDIAB), such as poly(p-phenylene benzimidazole). In an additional example, the rigid-rod polymer can include poly(phenylene terephthalate) (PPTA), such as poly(p-phenylene terephthalate). In another example, the rigid-rod polymer can include poly(benzimidazole) (ABPBI), such as poly(2,5(6)benzimidazole). In a further example, the rigid-rod polymer can include poly(benzoyl-1,4-phenylene-co-1,3-phenylene). In addition, the rigid-rod polymer can include any combination of the above copolymers. A particular rigid-rod polymer can include a polymer sold under the trademark PARMAX®, available from Mississippi Polymer Technology, Inc. of Bay St. Louis, Miss.

In addition, a particular rigid-rod polymer can be thermoplastic. In another example, a particular rigid-rod polymer can be dissolved in solvent. Such a rigid-rod polymer can be formed into complex shapes.

Further, a particular rigid-rod polymer can have a high crystallinity. For example, the rigid-rod polymer can have a crystallinity of at least about 30%, such as at least about 50%, or even, at least about 65%. Alternatively, the rigid-rod polymer can be amorphous.

A component of an implantable device can be formed of a polymeric material. In a particular example, the polymeric material can include a rigid-rod polymer. For example, the polymeric material can consist essentially of the rigid-rod polymer. In another example, the rigid-rod polymer can form a rigid-rod polymer matrix surrounding a filler. In a further example, the polymeric material can include a polymer blend.

In a particular example, the polymeric material can be substantially rigid-rod polymer, such as consisting essentially of rigid-rod polymer. In particular, the polymeric material can be a thermoplastic rigid-rod polymer absent or substantially free of filler.

In another example, the polymeric material can include a rigid-rod polymer matrix surrounding a filler. The filler can be a particulate filler, a fiber filler, or any combination thereof. In an example, the filler can include a ceramic, a metal, a carbon, a polymer, or any combination thereof. For example, the filler can include a ceramic, such as a ceramic oxide, a boride, a nitride, a carbide, or any combination thereof. In another example, the filler can include a metal, such as a particulate metal or metal fiber. An exemplary metal can include titanium, titanium alloy, tantalum, tantalum alloy, zirconium, zirconium alloy, stainless steel, cobalt, cobalt containing alloy, chromium containing alloy, indium tin oxide, silicon, magnesium containing alloy, aluminum, aluminum containing alloy, or any combination thereof. In another exemplary embodiment, the filler can include a carbon, such as carbon black, diamond, graphite, or any combination thereof. For example, a rigid-rod polymer matrix can be reinforced with a carbon fiber. In a further exemplary embodiment, the filler can include a polymer, such as a polymer particulate or a polymer fiber. The polymer can be, for example, a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. In an additional exemplary embodiment, the filler can include an agent, such as an agent absorbed in a carrier or a powdered agent.

In an exemplary embodiment, the polymeric material includes the rigid-rod polymer matrix and not greater than about 50 wt % of the filler. For example, the polymeric material can include not greater than about 30 wt % of the filler, such as not greater than about 15 wt % of the filler. Alternatively, the polymeric material can be substantially free of the filler.

In another exemplary embodiment, the polymeric material can be a polymer blend. For example, the polymer blend can be a homogeneous polymer blend in which a rigid-rod polymer and at least one other polymer form a single phase. In another example, the polymer blend can be a heterogeneous polymer blend in which a rigid-rod polymer and at least one other polymer form separate, yet intertwined phases. In particular, the polymer blend can include at least about 25 wt % of the rigid-rod polymer, such as at least about 30 wt %, at least about 50 wt % of the rigid-rod polymer, or even, at least about 75 wt % of the rigid-rod polymer. The at least one other polymer can be selected from a polyurethane material, a polyolefin material, a polystyrene, a polyurea, a polyamide, a polyaryletherketone (PAEK) material, a silicone material, a hydrogel material, a rigid-rod polymer, or any alloy, blend or copolymer thereof. Whether the blend is homogeneous or heterogeneous can depend on the selection of the rigid-rod polymer and the at least one other polymer, in addition to processing parameters and techniques.

In a particular exemplary embodiment, the polymer blend can be a heterogeneous blend in which the rigid-rod polymer is blended with a resorbable polymer, such as polylactic acid (PLA) or the like. Once implanted, the resorbable polymer may degrade or migrate leaving a rigid-rod polymer matrix having osteoconductive properties.

In another exemplary embodiment, the polymer blend can include a rigid-rod polymer blended with a second polymer to alter the modulus of the rigid-rod polymer. In a further exemplary embodiment, the polymer blend can include an agent, such as osteogenerative agent, a stimulating agent, a degradation agent, an analgesic, an anesthetic agent, an antiseptic agent, or any combination thereof. For example, the polymer blend can include the rigid-rod polymer and a hydrogel. The hydrogel can include an agent.

The polymer material including a rigid-rod polymer can have desirable physical and mechanical properties. For example, the polymer material can have a glass transition temperature of at least about 145° C., such as at least about 155° C., based on ASTM E1356.

In an example, the polymeric material can have an ultimate tensile strength at room temperature (23° C.) of at least about 125 MPa, such as at least about 135 MPa, at least about 150 MPa, at least about 180 MPa, or even, at least about 200 MPa, based on ASTM D638. In addition, the polymer material can exhibit an average tensile modulus at room temperature (23° C.) of at least about 5.0 GPa. For example, the polymer material can exhibit a tensile modulus of at least about 6.0 GPa, such as at least about 7.5 GPa. Further, the polymer material can have an elongation of about 1% to about 5%, such as about 2% to about 4%.

In a further example, the polymeric material including a rigid-rod polymer can exhibit a flexural yield strength at room temperature of at least about 220 MPa, such as at least about 250 MPa, or even at least about 300 MPa, based on ASTM D790. In addition, the polymeric material can exhibit a flexural modulus at room temperature (23° C.) of at least about 5.0 GPa, such as at least about 6.0 GPa, or even, at least about 7.5 GPa. Further, the polymeric material can exhibit a compressive yield strength at room temperature (23° C.) of at least about 250 MPa, such as at least about 300 NPa, or even, at least about 400 MPa, based on ASTM D695.

For a particular rigid-rod polymer, the mechanical properties of the polymeric material can be direction dependent. Alternatively, a particular rigid-rod polymer can provide a polymeric material having near isotropic mechanical properties, such as substantially isotropic mechanical properties.

Despite the strength of polymeric material including rigid-rod polymer, the polymeric material can have a low specific gravity. For example, the polymeric material can have a-specific gravity not greater than about 1.5, such as not greater than about 1.4, or even, not greater than about 1.3. Particular polymeric materials formed of a rigid-rod polymer can have a specific gravity not greater than about 1.27, or not greater than about 1.26, such as not greater than about 1.23 or even not greater than about 1.21, based on ASTM D792.

Further particular polymeric materials including rigid-rod polymer can exhibit low water absorption, such as a water hydration of not greater than 1.0% at equilibrium, based on ASTM D570. For example, the polymeric material can exhibit a water hydration not greater than about 0.7%, such as not greater than about 0.55%.

In a further example, polymeric materials including a rigid-rod polymer can form smooth surfaces, such as polished surfaces having low roughness (Ra). For example, the polymer material can form a surface having a roughness (Ra) not greater than about 100 nm. Particular polymeric materials including a rigid-rod polymer can form a surface having a roughness (Ra) not greater than about 10 nm, such as not greater than about 1.0 nm. In particular, a polymeric material formed of a rigid-rod polymer absent a filler can form a smooth surface. Such surfaces, can be used to form wear resistant surfaces that are subject to movement against an opposing surface, such as opposing surfaces of an intervertebral disc replacement. In another example, a polymeric material including a rigid-rod polymer in a polymer blend can form a smooth surface. Alternatively, the polymeric material can be roughened, shaped, or convoluted to form a rough surface. Such surfaces are particularly suited for engaging osteal structures, such as vertebrae.

In an additional embodiment, the polymeric material including a rigid-rod polymer can coat a metallic or ceramic article. For example, a rigid-rod polymer can coat a titanium component. In a particular example, a polymeric material including a rigid-rod polymer can be molded over a metallic component. Alternatively, the polymeric material including a rigid-rod polymer can be laminated to the metallic component, adhered to the metallic component, or mechanically fastened to the metallic component.

### Description of Agents

In an exemplary embodiment, an implantable device can include at least one reservoir, coating, or impregnated material configured to release an agent. The agent can generally affect a condition of proximate soft tissue, such as ligaments, a nucleus pulposus, an annulus fibrosis, or a zygapophysial joint, or can generally affect bone growth. For example, the agent can decrease the hydration level of the nucleus pulposus or can cause a degeneration of soft tissue, such as the nucleus pulposus, that leads to a reduction in hydration level, to a reduction in pressure, or to a reduction in size of, for example, the nucleus pulposus within the intervertebral disc. An agent causing a degeneration of soft tissue or a reduction in hydration level is herein termed a “degradation agent.” In another example, an agent can increase the hydration level of soft tissue, such as the nucleus pulposus, or can cause a regeneration of the soft tissue that results in an increase in hydration level or in an increase in pressure within the intervertebral disc, for example. Such an agent that can cause an increase in hydration or that can cause a regeneration of the soft tissue is herein termed a “regenerating agent.” In a further example, an agent (herein termed a “therapeutic agent”) can inhibit degradation of soft tissue or enhance maintenance of the soft tissue. Herein, therapeutic agents and regenerating agents are collectively referred to as “stimulating agents.” In a further example, an agent (e.g., an osteogenerative agent) can affect bone growth in proximity to the intervertebral disc or the zygapophysial joint. For example, an osteogenerative agent can be an osteoinductive agent, an osteoconductive agent, or any combination thereof.

An exemplary degradation agent can reduce hydration levels in the nucleus pulposus or can degrade the soft tissue, resulting in a reduction in hydration level or in pressure within the intervertebral disc, for example. For example, the degradation agent can be a nucleolytic agent that acts on portions of a nucleus pulposus. In an example, the nucleolytic agent is proteolytic, breaking down proteins.

An exemplary nucleolytic agent includes a chemonucleolysis agent, such as chymopapain, collagenase, chondroitinase, keratanase, human proteolytic enzymes, papaya protenase, or any combination thereof. An exemplary chondroitinase can include chondroitinase ABC, chondroitinase AC, chondroitinase ACII, chondroitinase ACIII, chondroitinase B, chondroitinase C, or the like, or any combination thereof. In another example, a keratanase can include endo-β-galactosidase derived from Escherichia freundii, endo-β-galactosidase derived from Pseudomonas sp. IFO-13309 strain, endo-β-galactosidase produced by Pseudomonas reptilivora, endo-β-N-acetylglucosaminidase derived from Bacillus sp. Ks36, endo-β-N-acetylglucosaminidase derived from Bacillus circulans KsT202,or the like, or any combination thereof. In a particular example, the degradation agent includes chymopapain. In another example, the degradation agent includes chondroitinase-ABC.

An exemplary regenerating agent includes a growth factor. The growth factor can be generally suited to promote the formation of tissues, especially of the type(s) naturally occurring as components of an intervertebral disc or of a zygapophysial joint. For example, the growth factor can promote the growth or viability of tissue or cell types occurring in the nucleus pulposus, such as nucleus pulposus cells or chondrocytes, as well as space filling cells, such as fibroblasts, or connective tissue cells, such as ligament or tendon cells. Alternatively or in addition, the growth factor can promote the growth or viability of tissue types occurring in the annulus fibrosis, as well as space filling cells, such as fibroblasts, or connective tissue cells, such as ligament or tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor P protein, bone morphogenetic protein, fibroblast growth factor, platelet-derived growth factor, insulin-like growth factor, or any combination thereof.

An exemplary therapeutic agent can include a soluble tumor necrosis factor α-receptor, a pegylated soluble tumor necrosis factor α-receptor, a monoclonal antibody, a polyclonal antibody, an antibody fragment, a COX-2 inhibitor, a metalloprotease inhibitor, a glutamate antagonist, a glial cell derived neurotrophic factor, a B2 receptor antagonist, a substance P receptor (NK1) antagonist, a downstream regulatory element antagonistic modulator (DREAM), iNOS, an inhibitor of tetrodotoxin (TTX)-resistant Na+-channel receptor subtypes PN3 and SNS2, an inhibitor of interleukin, a TNF binding protein, a dominant-negative TNF variant, Nanobodies™, a kinase inhibitor, or any combination thereof. Another exemplary therapeutic agent can include Adalimumab, Infliximab, Etanercept, Pegsunercept (PEG sTNF-R1), Onercept, Kineret200 , sTNF-R1, CDP-870, CDP-571, CNI-1493, RDP58, ISIS 104838, 1→3-β-D-glucan, Lenercept, PEG-sTNFRII Fc Mutein, D2E7, Afelimomab, AMG 108, 6-methoxy-2-napthylacetic acid or betamethasone, capsaiein, civanide, TNFRc, ISIS2302 and GI 129471, integrin antagonist, alpha-4 beta-7 integrin antagonist, cell adhesion inhibitor, interferon gamma antagonist, CTLA4-Ig agonist/antagonist (BMS-188667), CD40 ligand antagonist, Humanized anti-IL-6 mAb (MRA, Tocilizumab, Chugai), HMGB-1 mAb (Critical Therapeutics Inc.), anti-IL2R antibody (daclizumab, basilicimab), ABX (anti IL-8 antibody), recombinant human IL-1 0, HuMax IL-15 (anti-IL 15 antibody), or any combination thereof.

An osteogenerative agent, for example, can encourage the formation of new bone (“osteogenesis”), such as through inducing bone growth (“osteoinductivity”) or by providing a structure onto which bone can grow (“osteoconductivity”). Generally, osteoconductivity refers to structures supporting the attachment of new osteoblasts and osteoprogenitor cells. As such, the agent can form an interconnected structure through which new cells can migrate and new vessels can form. Osteoinductivity typically refers to the ability of the implantable device or a surface or a portion thereof to induce nondifferentiated stem cells or osteoprogenitor cells to differentiate into osteoblasts.

In an example, an osteoconductive agent can provide a favorable scaffolding for vascular ingress, cellular infiltration and attachment, cartilage formation, calcified tissue deposition, or any combination thereof. An exemplary osteoconductive agent includes collagen; a calcium phosphate, such as hydroxyapatite, tricalcium phosphate, or fluorapatite; demineralized bone matrix; or any combination thereof.

In another example, an osteoinductive agent can include bone morphogenetic proteins (BMP, e.g., rhBMP-2); demineralized bone matrix; transforming growth factors (TGF, e.g., TGF-β); osteoblast cells, growth and differentiation factor (GDF), LIM mineralized protein (LMP), platelet derived growth factor (PDGF), insulin-like growth factor (ILGF), or any combination thereof. In a further example, an osteoinductive agent can include HMG-CoA reductase inhibitors, such as a member of the statin family, such as lovastatin, simvastatin, pravastatin, fluvastatin, atorvastatin, cerivastatin, mevastatin, pharmaceutically acceptable salts esters or lactones thereof, or any combination thereof. With regard to lovastatin, the substance can be either the acid form or the lactone form or a combination of both. In a particular example, the osteoinductive agent includes a growth factor. In addition, osteoconductive and osteoinductive properties can be provided by bone marrow, blood plasma, or morselized bone of the patient, or other commercially available materials.

In addition, other agents can be incorporated into a reservoir, such as an antibiotic, an analgesic, an anti-inflammatory agent, an anesthetic, a radiographic agent, or any combination thereof. For example, a pain medication can be incorporated within a reservoir or a release material in which another agent is included or can be incorporated in a separate reservoir or release material. An exemplary pain medication includes codeine, propoxyphene, hydrocodone, oxycodone, or any combination thereof. In a further example, an antiseptic agent can be incorporated within a reservoir. For example, the antiseptic agent can include an antibiotic agent. In an additional example, a radiographic agent can be incorporated into a reservoir, such as an agent responsive to x-rays.

Each of the agents or a combination of agents can be maintained in liquid, gel, paste, slurry, solid form, or any combination thereof. Solid forms include powder, granules, microspheres, miniature rods, or embedded in a matrix or binder material, or any combination thereof. In an example, fluids or water from surrounding tissues can be absorbed by the device and placed in contact with an agent in solid form prior to release. Further, a stabilizer or a preservative can be included with the agent to prolong activity of the agent.

In particular, one or more agents can be incorporated into a polymeric matrix, such as a hydrogel, a bioresorbable polymer, or a natural polymer. An exemplary hydrogel can include polyacrylamide (PAAM), poly-N-isopropylacrylamine (PNIPAM), polyvinyl-methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or any combination thereof. An exemplary bioresorbable polymer can include polylactide (PLA), polyglycolide (PGA), poly(lactide-co-glycolide) (PLGA), polyanhydride, polyorthoester, or any combination thereof. An exemplary natural polymer can include a polysaccharide, collagen, silk, elastin, keratin, albumin, fibrin, or any combination thereof.

### Embodiments of Implantable Device

According to an aspect, an implantable device can include a first component configured to fixably attach to a first vertebra to secure the first vertebra in a position relative to a second vertebra. The component can include a polymeric material including a rigid-rod polymer. In general, the implantable devices provided herein can be implanted proximate to the spinal column, such as near or around the spinal column and more particularly, fixably attached to the spinal column. For clarity, the terms “spinal column” or “spine” as used herein, refers to all portions of the spine, including the bones, discs, muscles, and ligaments unless otherwise stated. Moreover, the components provided herein can include fixably attaching components that can engage the spine to limit movement of the component(s) relative to a portion of the spine.

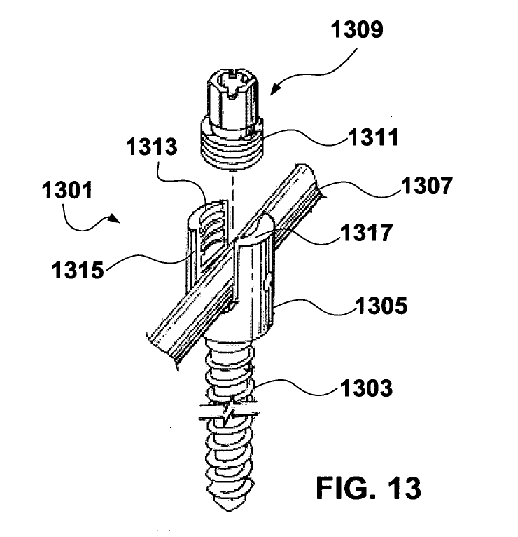

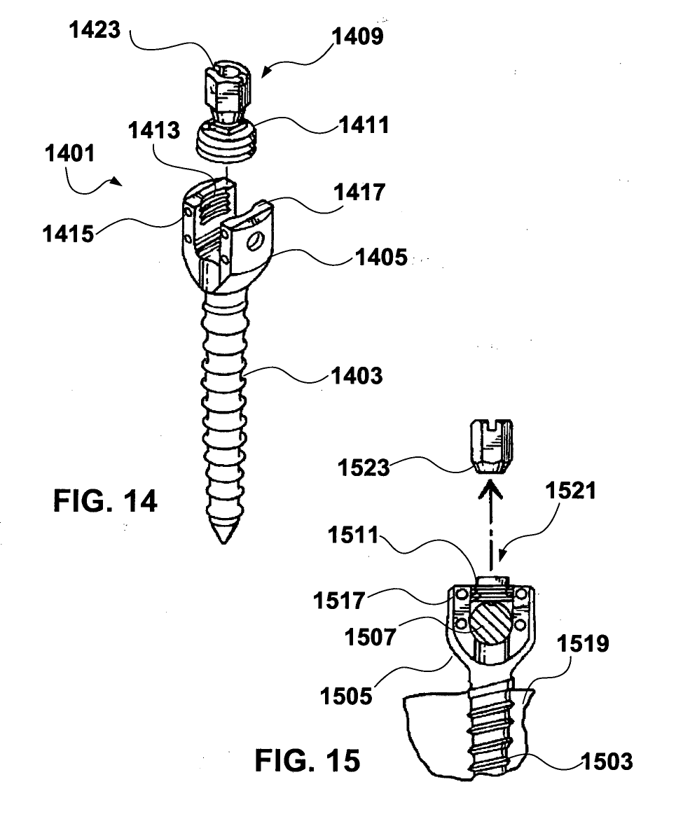

According to an embodiment, the component can be a single component, such as a component configured to fixably attach to a first vertebra and a second vertebra. The component can be attached to the exterior of the vertebra, such as along the surface of a vertebra, as a coupling component, or alternatively, the component can be affixed to bone as a load bearing component. Referring to FIG. 6, a rod 601 is illustrated that can be configured to fixably engage the spine, or a portion of the spine. FIG. 7 also illustrates a rod 701, having threads 703. The threads 703 can be used to support other implantable devices, such as fasteners, to fixably engage the rod 701 with a portion of the spine. In a particular example, a polymeric material including a rigid-rod polymer can form at least a portion of the rod 601 or the rod 701.

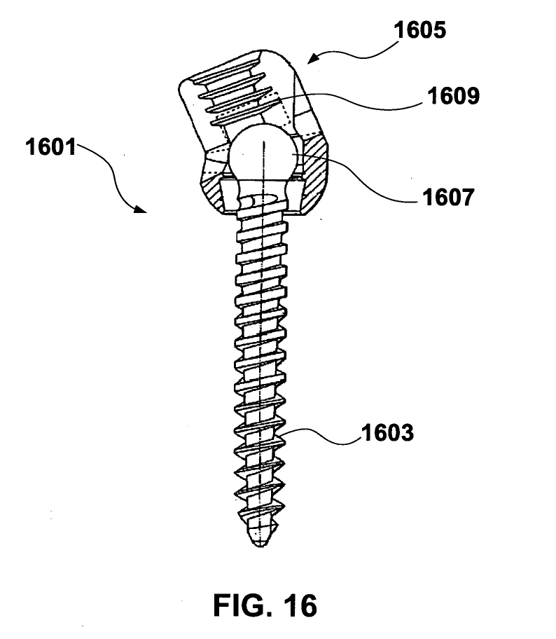

Referring to FIG. 8, according to another embodiment, a rod 801 can include threads 803, as well as a break region 805 defined between scored sections 807 and 809. According to a particular embodiment, the rod 801 can be scored using a tool and broken to accommodate a desired length. Such a procedure is suitable outside of a patient or while operating in vivo. The rod 801 can be made of a polymeric material including a rigid-rod polymer, and according to a particular embodiment, the rod 801 can consist essentially of a rigid-rod polymer material, absent fillers and additives. Such rigid-rod polymer materials can provide a suitable combination of weight and brittleness, making the breaking procedure easy and providing a clean break surface without significant distortion of the threads.

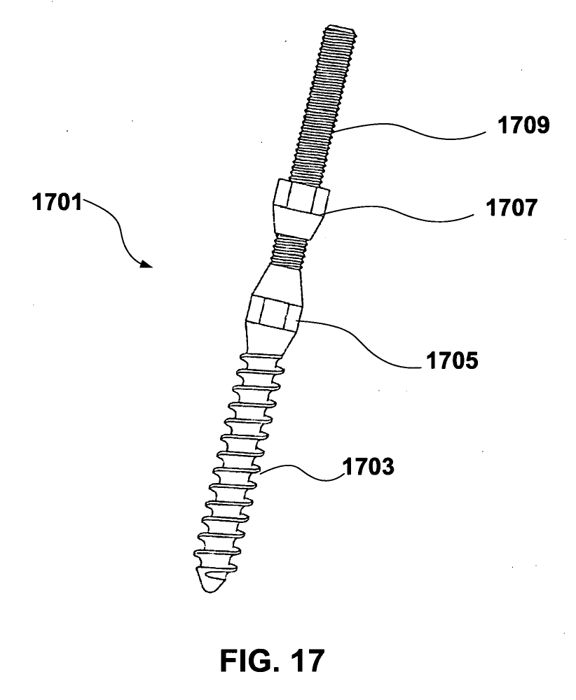

FIG. 9 illustrates another embodiment of a rod 901, having threaded portions 903 and 905, and also having a movable portion 907, which can be used to change the direction of a portion of the rod 901. The movable portion 907 can include, for example, a locking hinge suitable for changing the direction of the rod 901. In another embodiment, the movable portion 907 can include a sleeve of pliable material, such as a metal or a polymer. While not illustrated, any of the above illustrated rods 601, 701, 801, and 901, can be curved or bent. In a particular example, the rod 901 or a portion thereof, such as the movable portion 907, can be formed of a polymeric material including a rigid-rod polymer.

According to another embodiment, a component of the implantable device can include a fastener. Generally, fasteners can be configured to fixably attach a plate or rod to other particular parts of the body, such as bones, like the vertebrae. According to embodiments herein, fasteners can include screws, bolts, pegs, nuts, hooks, or the like. According to an embodiment, the components provided herein, particularly the fasteners, can be configured to fixably engage a portion of the spine, such as a vertebra, a spinous process, or a plurality of the like. In a particular embodiment, the component can be configured to be located, at least partially, in an intervertebral space, or in a facet body.

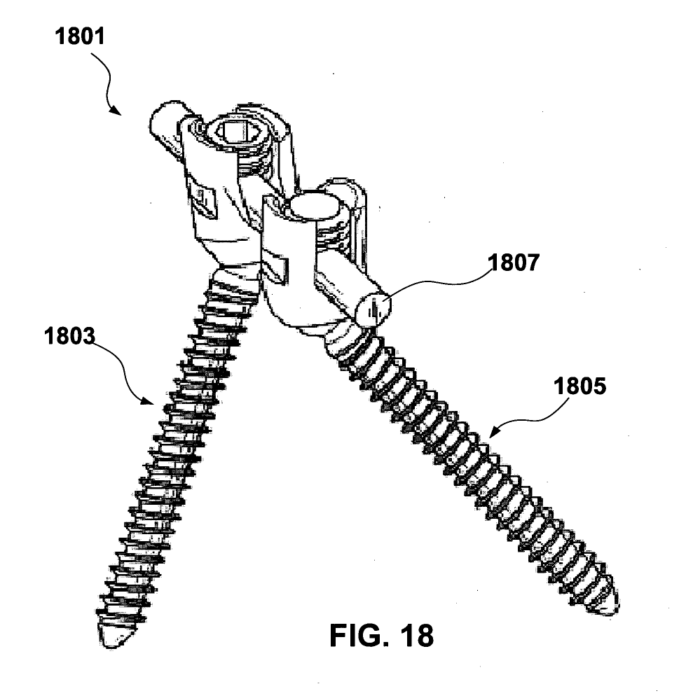

Referring to FIG. 10, a bolt 1001 is illustrated having threads 1003 and a head 1005. According to an embodiment, the bolt threads 1003 can include a polymeric material including a rigid-rod polymer. In a particular embodiment, the entire bolt 1001, including the threads 1003 and head 1005 can include a polymeric material including a rigid-rod polymer. While a hex-head bolt is illustrated, it will be appreciated that other types of bolts can be used.

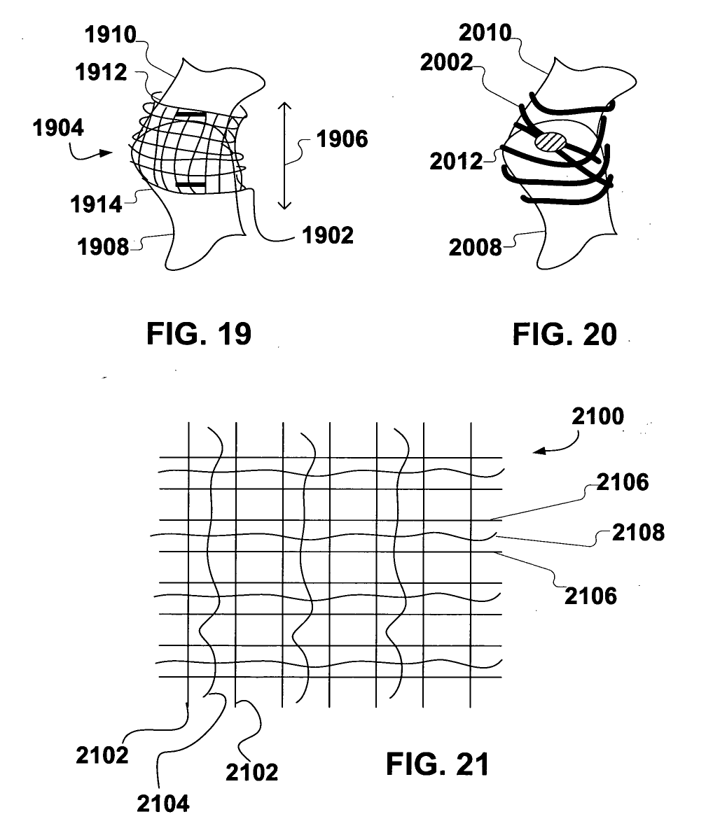

According to another embodiment, the fastener can be a screw 1101, as illustrated in FIG. 11. In an example, the screw 1101 can include a polymeric material including a rigid-rod polymer. For example, the threads 1103, or the head 1105, or both, can include the polymeric material. Suitable various modifications of the screw 1101, among others, can include pedicle screws, polyaxial screws, or any combination thereof.

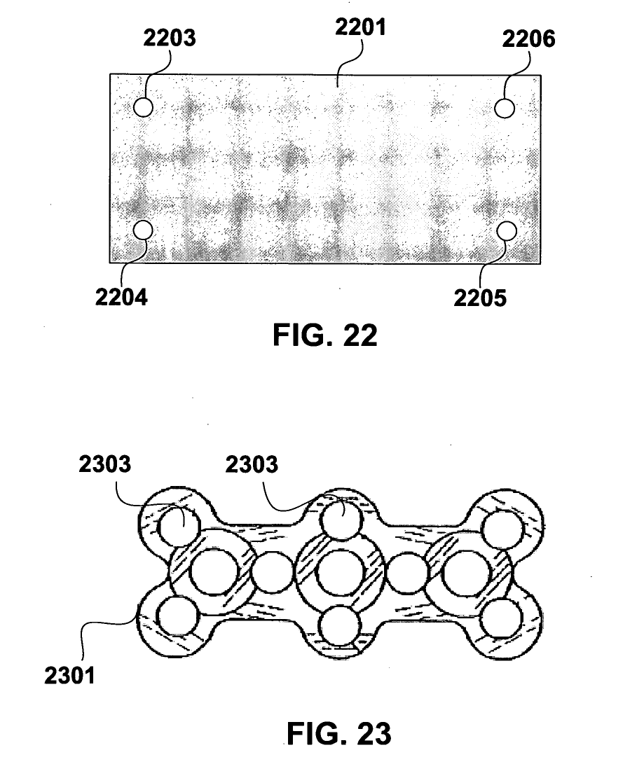

According to another embodiment illustrated in FIG. 12, the fastener can include a screw 1201. The screw 1201 can be configured to fixably engage certain portions of the spine. For example, a screw 1201 can be configured to be located at least partially in a facet space between two vertebrae. In an example, the screw 1201 can have threads 1203 and a head 1205, and also can incorporate a coupling head 1207 for coupling the screw 1201 to a rod 1213. The coupling head 1207 can be fixed onto a normal screw (as illustrated in FIG. 11) or can be an integrated portion of a more complex screw arrangement (FIG. 12). The coupling head 1207 can include winged portions 1209 and 1211 having openings (not illustrated) through which a rod 1213 can be fed. The rod 1213 and the screw 1201 can interlock and be rigidly fixed relative to each other and relative to portions of the spine. In an embodiment where the rod to be engaged is a threaded rod, the openings in the winged portions 1209 and 1211 can be threaded to engage the threads of the rod 1213. Further, the coupling head 1207 or the winged portions 1209 and 1211 can include a polymeric material including a rigid-rod polymer.

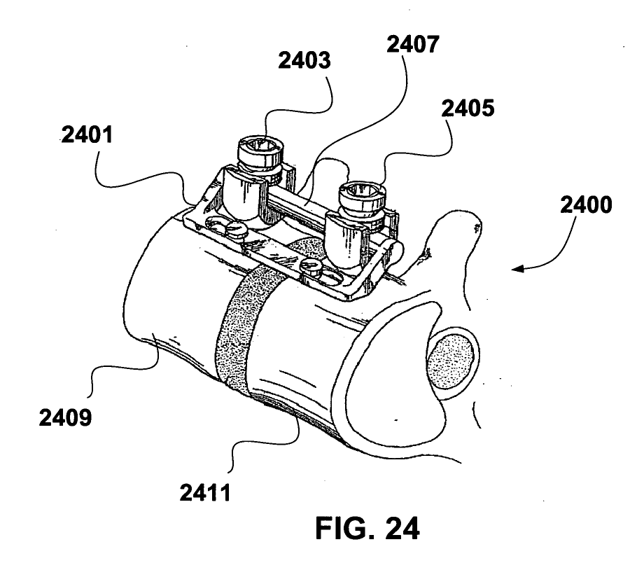

Referring to FIG. 13, a perspective view of a screw 1301 engaging a rod 1307 is illustrated. According to the illustrated embodiment, the screw 1301 can include a threaded portion 1303 and a coupling head 1305 for receiving the rod 1307. Unlike the previous embodiment illustrated in FIG. 12, the rod 1307 can be engaged between the winged portions 1315 and 1317, instead of through openings in the winged portions. Accordingly, the screw 1301 can include a retaining portion 1309, which includes a threaded portion 1311 for engaging a threaded portion 1313 in the body of the coupling head 1305. The retaining portion 1309 can be screwed into the coupling head 1305 after the rod 1307 is engaged to fixably attach the screw 1301 and the rod 1307 relative to each other. Likewise, the components of this particular embodiment of the screw 1301 can include a polymeric material including a rigid-rod polymer. According to a particular embodiment, each of the components of the screw 1301 can be formed essentially of a rigid-rod polymer material.

Referring to FIG. 14 and FIG. 15, a perspective view of a screw 1401 similar to the screw of FIG. 13 is illustrated. As described previously, the screw 1401 can have a coupling head 1405 having multiple components, notably a retaining portion 1409 for screwing into the coupling head 1405 and retaining a rod. In a particular embodiment, the retaining portion 1409 can include a head portion 1423 configured to receive a fastening tool, such as a screwdriver or the like for engaging the retaining portion 1409 with the coupling head 1405. Referring to FIG. 15, a screw 1501 is illustrated as engaged in a surface 1519. According to an embodiment, the head portion 1523 can be separated from the coupling head 1505 at a break region 1521. According to a particular embodiment, the retaining portion 1511 and the head portion 1523 can be formed essentially of a rigid-rod polymer material, which can facilitate suitable breaking with minimal backlash or minimal elongation.

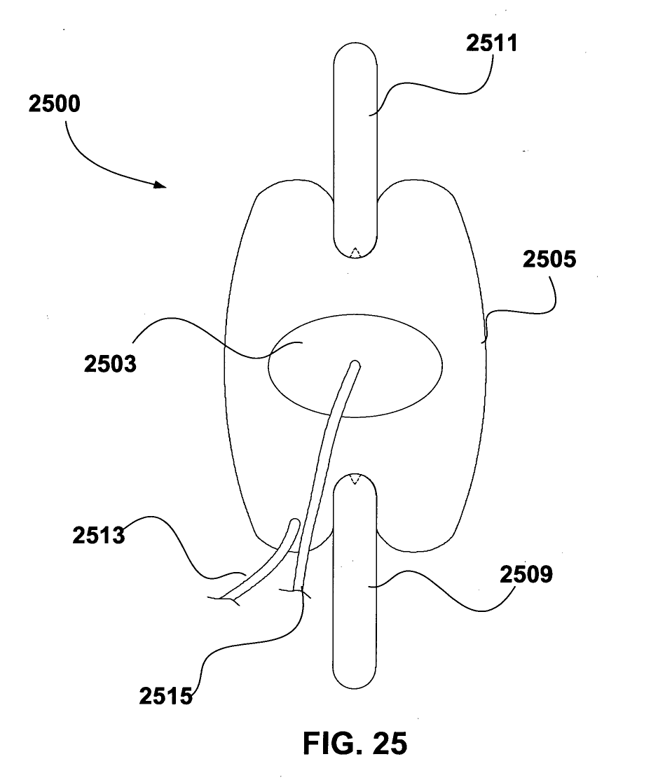

In continued reference to fasteners, FIG. 16 illustrates an embodiment of a polyaxial screw 1601 having a threaded portion 1603 and a head 1605. The head 1605 houses a ball structure 1607 which facilitates hemispherical motion of the head 1605 relative to the threaded portion 1603 and allows for variable attachment angles as well as variable fastening angles during a procedure. The head 1605 also houses a threaded portion 1609 for engaging a variety of fastening tools, as well as other threaded structures, including a threaded rod. In accordance with previous embodiments, each of the components of the polyaxial screw 1601 can be made of a polymeric material including a rigid-rod polymer.

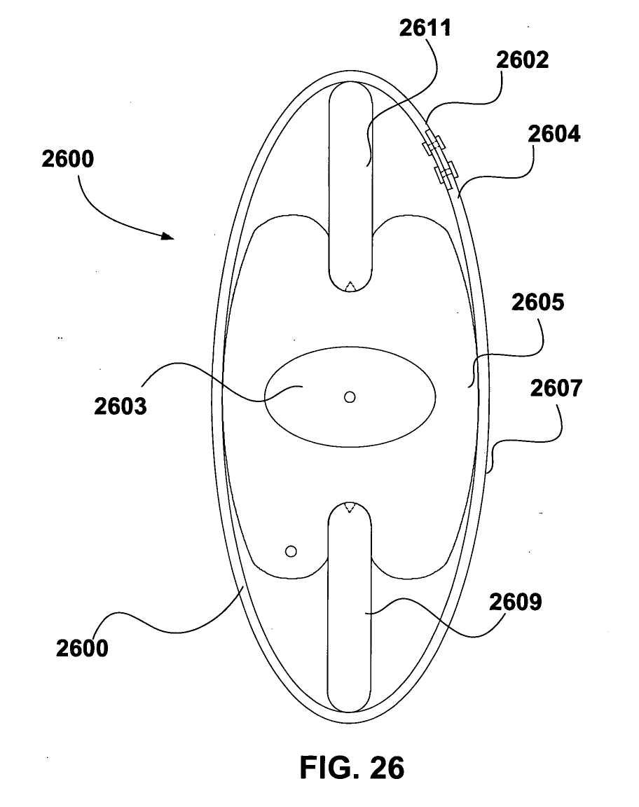

Referring to FIG. 17, a pedicle screw 1701 is illustrated. According to an embodiment, the pedicle screw 1701 includes threads 1703 and a coupling head 1705, which is attached to an extension portion 1707, which is configured to receive a threaded rod 1709. The pedicle screw 1701 can be inserted into a pedicle of a vertebra and can be used to support an assembly, which can include other screws and plates or rods to immobilize a portion of the spine. In a particular example, the pedicle screw or a portion thereof can be formed of a polymeric material including a rigid-rod polymer.

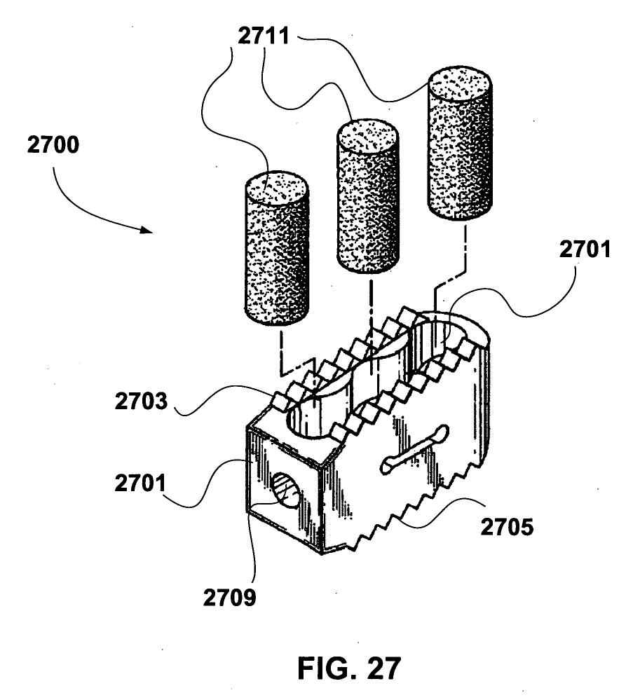

Referring to FIG. 18, an assembly 1801 is illustrated that includes a rod 1807 fixably attached to a plurality of screws 1803 and 1805. According to a particular embodiment, the implantable device can include a plurality of components, such as rods and screws, interlocked and fixably attached relative to one another. In a particular embodiment, the screws 1803 and 1805 are polyaxial screws which facilitate fixably attaching the assembly 1801 to a plurality of bones and positions along the spine. In accordance with previous embodiments, a portion of a component can be formed of a polymeric material including a rigid-rod polymer.

According to another embodiment, the component can include a mesh or strand material. For example, a mesh material can be wrapped around a portion of the spine for stabilization, such as around the zygapophysial joint and secured to the inferior or the superior articular processes associated with the zygapophysial joint. In a particular embodiment, a strand material can be wrapped around the articular processes associated with the zygapophysial joint and secured to itself.

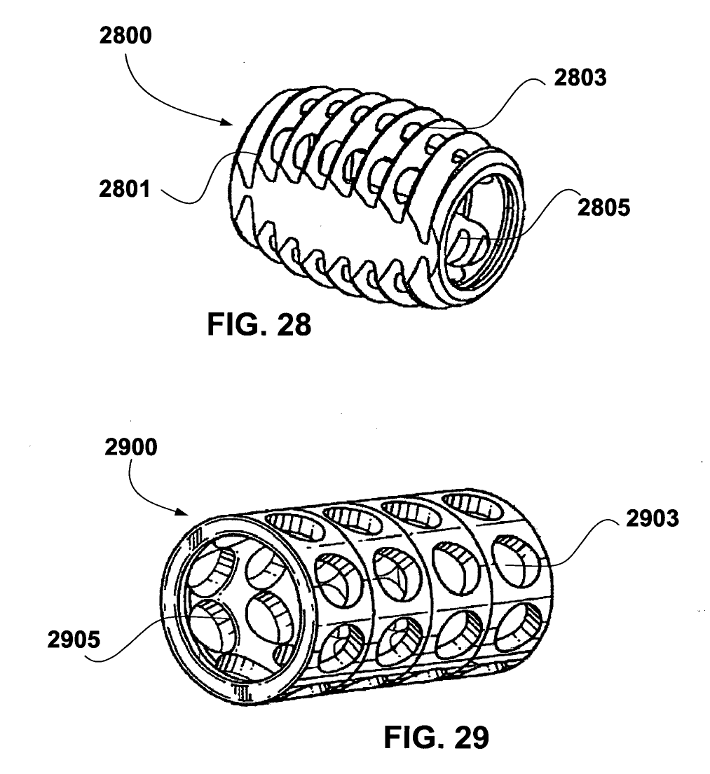

FIG. 19 includes an illustration of a mesh material 1902 wrapped around a zygapophysial joint 1904. The zygapophysial joint 1904 is formed between a superior articular process 1908 of an inferior vertebra and an inferior articular process 1910 of a superior vertebra. The directional indicator 1906 indicates the general axis of the spine formed by the vertebrae of which the processes 1908 and 1910 are part. As illustrated, the mesh material 1902 can be secured to the inferior articular process 1910 via a fastener 1912 and to the superior articular process 1908 via a fastener 1914. Alternatively, the mesh material 1902 can be secured to itself via the fastener 1912 or the fastener 1914. The fasteners 1912 and 1914 can include a screw, a staple, a crimp fastener, or any combination thereof. Accordingly, the components of the mesh, particularly the mesh material 1902 and the fasteners 1912 and 1914 can include a polymeric material including a rigid-rod polymer. In a particular embodiment, the mesh 1902 can include a rigid rod polymer combined with a flexible material, such as an elastomer, or the like. In a further example, the mesh 1902 can be formed of hydrogel material blended with a polymeric material including a rigid-rod polymer. The hydrogel material can include an agent.

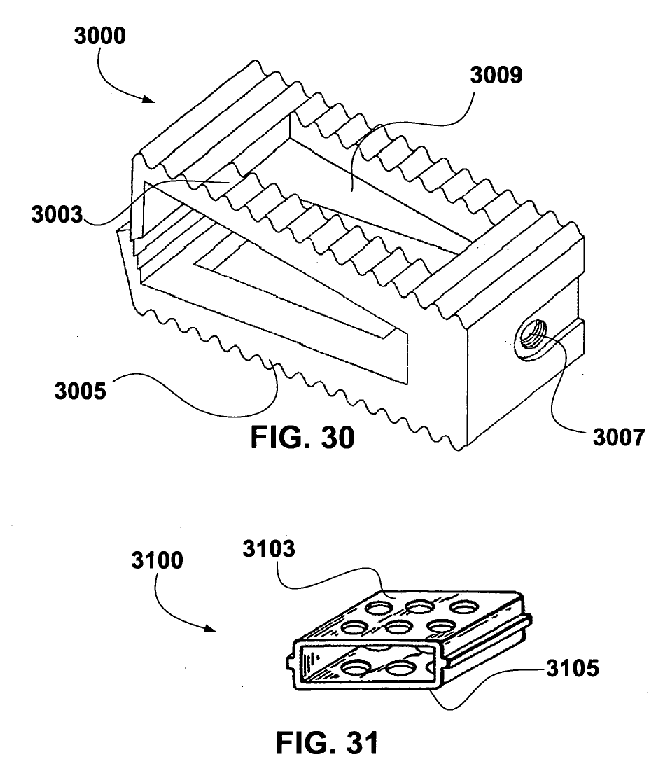

In another embodiment, FIG. 20 includes an illustration of an exemplary strand material 2002 wrapped around a zygapophysial joint 2004. The zygapophysial joint 2004 is formed between a superior articular process 2008 of an inferior vertebra and an inferior articular process 2010 of a superior vertebra. The strand material 2002 can engage the inferior articular process 2010 and the superior articular process 2008 by being wrapped around the processes 2008 and 2010. The strand material 2002 can be secured to itself by the fastener 2012. As described in accordance with embodiments herein, the strand material 2002 and the fastener 2012 can include a polymeric material including a rigid-rod polymer. It will also be appreciated that the mesh material or the strand material provided in previous embodiments, can be attached to various portions of the spine.

In another embodiment, the mesh material can include a sheet of strands interwoven together or secured together with a coating. FIG. 21 includes an illustration of an exemplary mesh material 2100 including interwoven strands 2102, 2104, 2106, and 2108. In an example, the interwoven strand 2102 represents a warp strand and the interwoven strand 2106 represents a weft strand. Generally, the warp strand 2102 can include a rigid-rod polymer material. According to an embodiment, both the warp strand 2102 and the weft strand 2106 include a rigid-rod polymer material.

In the example illustrated in FIG. 21, the warp strands 2102 and the weft strands 2106 form a substantially orthogonal pattern, forming approximately 90° angles at the intersection between the warp strands 2102 and the weft strands 2106. When installed in a patient, the mesh material can be positioned such that the warp strands 2102 align with the general axis of an upright spine. In another example, the weft strand 2106 can align with the general axis. Still, in an alternative embodiment, the strands can intersect to form acute angles. In an exemplary embodiment, the acute angle a can be not greater than about 65°, such as not greater than about 45°. In a particular example, the mesh material can be installed such that a bisection of the angle a can align with the general axis. Alternatively, the warp strands 2102 or the weft strands 2106 can be aligned with the general axis.

In a further exemplary embodiment, the mesh material can include an agent, such as a stimulating agent, a degradation agent, an osteogenerative agent, an anesthetic agent, or any combination thereof. In an example, the agent can be included in a controlled release material incorporated into the mesh material. In another example, the mesh material can be configured to enclose the agent, holding the agent in proximity to a desired location. In a further example, the mesh material can be coated in a release material.

As illustrated in FIG. 21, the mesh material 2100 can also include interwoven strands that include an agent. For example, the mesh material 2100 can include warp strands 2102 and weft strands 2106 forming an interwoven material. In an example, a controlled release strand 2104 can be included between the warp strands 2102. In another example, a controlled release strand 2108 can be included between the weft strands 2106. In an exemplary embodiment, the controlled release strands (2104 and 2108) can be formed of controlled release materials. For example, the controlled release strands (2104 and 2108) can be formed of hydrogel materials. In another example, the controlled release strands (2104 and 2108) can include a coating including the agent. For example, the coating can include a slow dissolving solid matrix that releases the agent as it dissolves

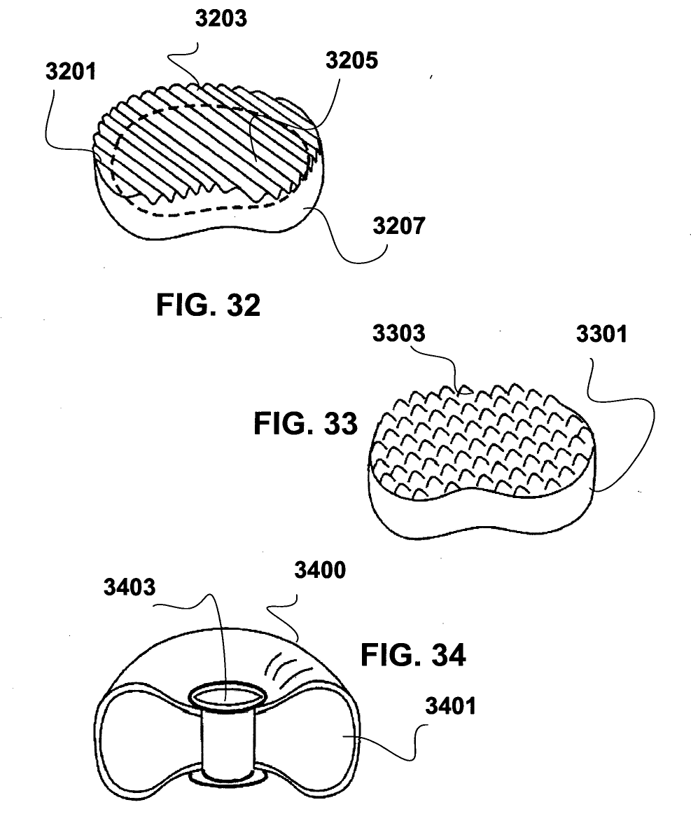

In further reference to various components that can be used, FIG. 22 illustrates a plate 2201 that can be inserted to fixably attach to the spine. Generally, plates can be used in various locations, such as in the cervical, lumbar, or sacral region of the spine, typically between an upper and lower vertebrae to interlock and immobilize the vertebrae. The plate 2201 can have a plurality of holes 2203, 2204, 2205, and 2206 for receiving screws to fixably attach the plate to a portion of the spine.

Referring to FIG. 23, another plate 2301 is illustrated according to a further embodiment. The plate 2301 has an intricate shape and can have a contour to fit particular locations along the spine. Like plate 2201, plate 2301 can have a plurality of holes 2303 within the frame to receive fasteners for affixing the plate to the spine. A suitable plate material can include a polymeric material including a rigid-rod polymer, which can be suitably rigid and has substantially isotropic mechanical properties to withstand different types of forces including multi-axial loads and torque.

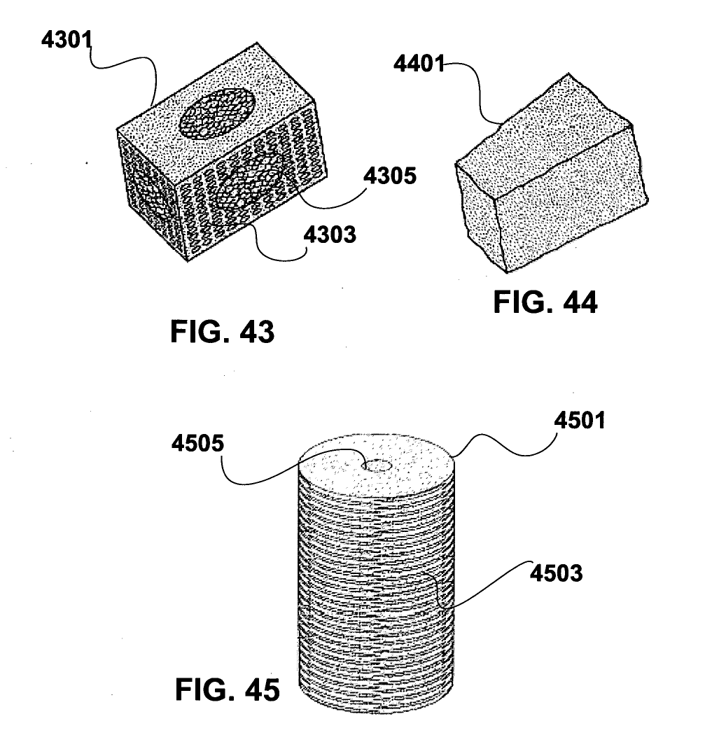

Referring to FIG. 40, an exemplary lumbar plate 4001 is illustrated. The lumbar plate 4001 can have a curvature to fixably engage the vertebrae of the lumbar region. As illustrated in previous embodiments, the lumbar plate 4001 can have holes 4003 and 4005 for receiving screws for fixing the lumbar plate 4001 to the appropriate vertebrae. Referring to FIG. 41, a rear view of the lumbar plate 4101 is illustrated, particularly to illustrate gripping components 4103 and 4105 at the ends of the plate to frictionally engage the plate with the adjacent vertebrae and provide superior stabilization of the vertebrae.

FIG. 42 illustrates a lumbar plate 4201 engaging an upper lumbar vertebrae 4203 and a lower lumbar vertebrae 4205. Generally, the lumber plate 4201 can be placed in an anterior position with regard to the spine in order to fix the upper and lower vertebrae 4203 and 4205 relative to each other as well as stabilize the intervertebral disc 4207, or a prosthetic that may be placed in the intervertebral disc space. As illustrated, the lumbar plate 4201 can engage the upper and lower vertebrae 4203 and 4205 using fastening devices or the like. According to an embodiment, and as illustrated in FIG. 42, the fastening devices can include upper and lower screws 4209 and 4211 respectively.

As discussed above, suitable materials for plates can include polymers, such as rigid-rod polymers. According to one embodiment, the plate can include essentially a rigid-rod polymer material, such as a homogenous rigid-rod polymer matrix, essentially free of fillers. Such materials provide a suitable combination of mechanical properties such as, for example, rigidity and flexural modulus that more aptly mimics the properties of bone. In contrast to traditional metallic and ceramic components, use of such materials results in less stress shielding of the surrounding tissue and bone. Stress shielding can cause a decrease in bone growth and the integrity of bone that grows. In particular, a polymeric material including a rigid-rod polymer can provide the component with an improved strength over traditional polymeric materials, while providing a reduced modulus to that of metallic or ceramic material, providing both sufficient support while preventing stress shielding. The effects of stress shielding are typically a result of using conventional materials such as titanium or other polymers, including heterogenous, reinforced polymer materials.

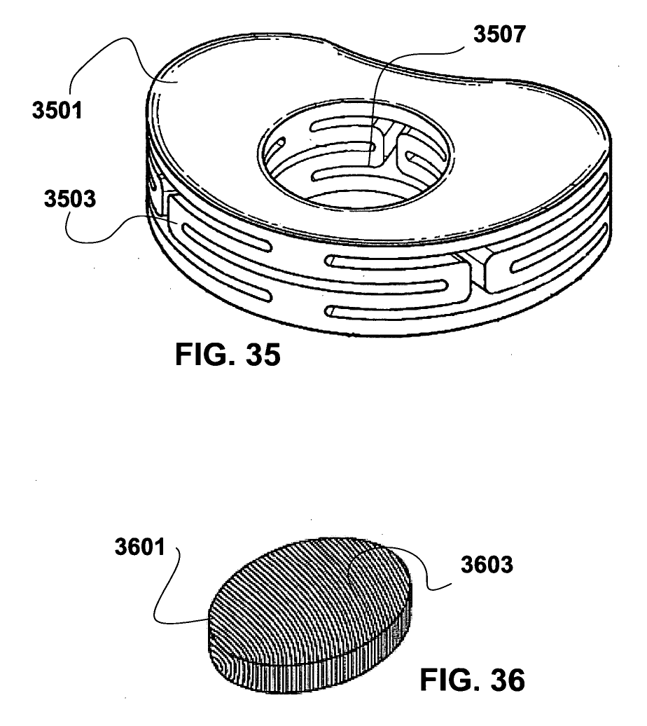

In reference to FIG. 24, a stabilization system 2400 is illustrated. According to an embodiment, the components herein can be used in combination to form more complex stabilization systems. Accordingly, a stabilization system 2400 can include a plate 2401 in conjunction with fasteners 2403 and 2405, and a rod 2407 to stabilize two vertebrae 2409 and 2411 and secure the vertebrae in a position relative to each other. A suitable material for one or more of the components can include a polymeric material including a rigid-rod polymer.

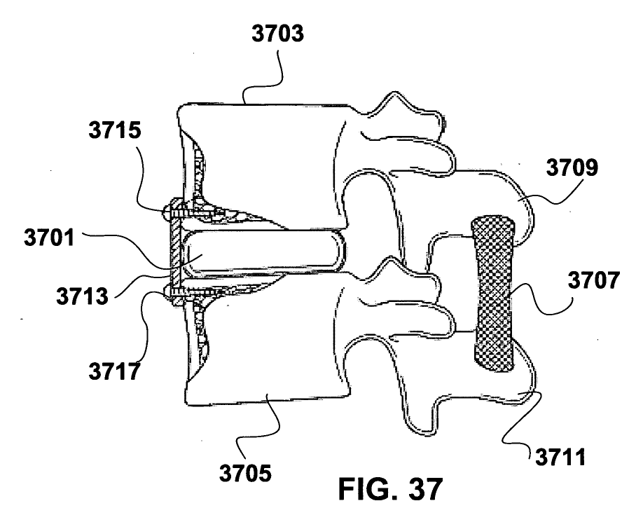

In further reference to complex components that can be used in various locations around the spine, a component can be configured to engage two spinous processes where one spinous process is associated with a superior vertebra and the other spinous process is typically associated with an inferior vertebra. Referring to FIG. 25, an interspinous process brace 2500 is illustrated according to an embodiment. The interspinous process brace 2500 can be an expandable component and can comprise an outer chamber 2505 and an inner chamber 2503, which can be expanded to engage the inferior and superior spinous processes 2509 and 2511. Various methods can be used to expand the brace 2500. In an example, expanding the brace 2500 can include injecting a curable polymer, pneumatically expanding a balloon, or engaging rigid and slidably engageable components that can be expanded using a wrench or fastening tool. In the illustrated embodiment of FIG. 25, the interspinous process brace can be expanded, e.g., by injecting one or more materials into the chambers 2503, 2505, in order to increase the distance between the superior spinous process 2511 and the inferior spinous process 2509.

Alternatively, a distractor can be used to increase the distance between the two processes and the expandable interspinous process brace 2500 can be expanded to support the two processes. After the expandable interspinous process brace 2500 is expanded accordingly, the distractor can be removed and the expandable interspinous process brace 2500 can support the two processes to substantially prevent the distance between the superior spinous process 2511 and the inferior spinous process 2509 from returning to a pre-distraction value.

In a particular embodiment, the multi-chamber expandable interspinous process brace 2500 can be injected with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethanes, polyolefins, silicones, silicone polyurethane copolymers, polymethylmethacrylate (PMMA), epoxies, cyanoacrylate, hydrogels, or a combination thereof. Further, the polyolefin materials can include polypropylenes, polyethylenes, halogenated polyolefins, or flouropolyolefins. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly(2-ethyl)oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. In a particular example, a ceramic can be included, such as calcium phosphate, hydroxyapatite, calcium sulfate, bioactive glass, or a combination thereof. In an alternative embodiment, the injectable biocompatible materials can include one or more fluids such as sterile water, saline, or sterile air.

In a particular embodiment, portions of the chambers 2503 or 2505 can be formed of a polymeric material including a rigid-rod polymer. For example, surface portions of 2505 that engage the spinous processes can be formed of the polymeric material including a rigid-rod polymer. In an alternative embodiment, the chambers can be replaced with rigid components formed of a polymeric material including a rigid-rod polymer.

FIG. 26 illustrates a further embodiment of an interspinous process brace 2600 that includes a tether 2607 which can be installed around the interspinous process brace 2600. As shown, the tether 2607 can include a proximal end 2602 and a distal end 2604. In a particular embodiment, the tether 2607 can circumscribe the expandable interspinous process brace 2600 and the spinous processes 2611, 2609. Further, the ends 2602, 2604 of the tether 2607 can be brought together and one or more fasteners can be installed therethrough. In an example, the one or more fasteners can be formed of a polymeric material including a rigid-rod polymer. Accordingly, the tether 2607 can be installed in order to prevent the distance between the spinous processes 2609, 2611 from substantially increasing beyond the distance provided by the interspinous process brace 2600.

In a particular embodiment, the tether 2607 can comprise a biocompatible material that flexes during installation and provides a resistance fit against the inferior process. Further, the tether 2607 can comprise a substantially non-resorbable suture or the like. According to another embodiment, the tether 2607 can include a rigid-rod polymer material, particularly, a rigid-rod polymer material incorporating an elastic material for suitable elasticity and rigidity.

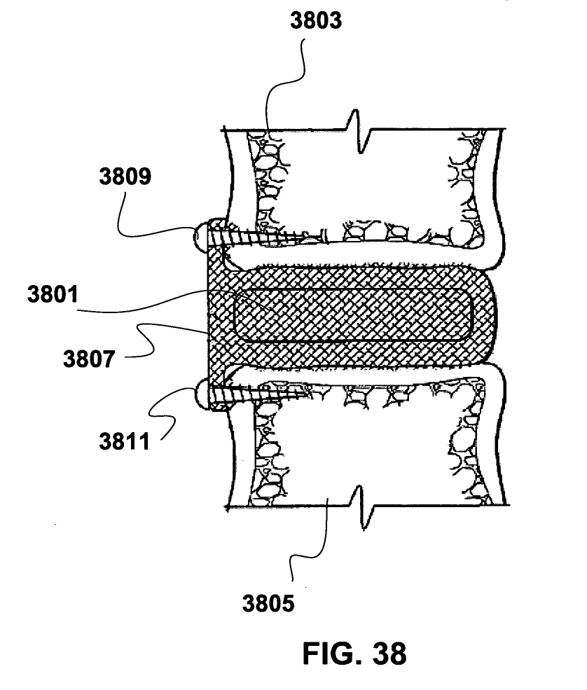

Referring to FIG. 27, a fusion cage 2700 is illustrated. Generally, fusion cages can be provided in an intervertebral space in the place of a disc that has been previously removed. Typically, fusion cages can have a simple geometrical contour, such as a rectangular contour, a spherical or cylindrical contour, a frusto-conical contour, a conical contour, a disc-like contour, or the like. In addition to the shape, according to an embodiment, the fusion cage 2700 can have an engagement surface with surface features for frictionally engaging a portion or portions of a vertebra, which, in the context of fusion cages, are generally the vertebral bodies of the superior and inferior vertebrae. Referring to FIG. 27, the fusion cage 2700 can have teeth on the top surface 2703 and the bottom surface 2705 to frictionally engage the vertebrae and hold the fusion cage in the intervertebral disc space between the vertebrae.

In addition to the shape, fusion cages, such as the fusion cage 2700, can be porous. According to another embodiment, the fusion cage can be hollow. Referring to FIG.27, the illustrated fusion cage 2700 is a generally hollow component, having a chamber 2707 for receiving sponges 2711. In addition to the chamber 2707, the end 2701 of the fusion cage 2700 can have an aperture 2709. The porous, often hollow design of the fusion cage 2700 can facilitate delivery of a bioactive agent that can be used to facilitate bone growth. The bioactive agent can be an osteogenerative agent (described above), which according to an embodiment, can be provided within or on the surface of a fusion cage. According to the embodiment illustrated in FIG. 27, sponges 2711 can be inserted into the chamber 2707, the sponges 2711 can be soaked in an osteogenerative agent. A suitable material for the fusion cage 2700 can include a rigid-rod polymer material that is strong and resistant to multi-axial forces including axial loading and torque. In a further embodiment, the fusion cage 2700 can include a polymeric material including a rigid-rod polymer. In an example, the polymeric material is a polymer blend including the rigid-rod polymer and a resorbable polymer.

Other exemplary embodiments of fusion cages are illustrated in FIGS. 28-31. Referring briefly to the cages illustrated in FIG. 28 and FIG. 29, the fusion cages 2800 and 2900, respectively, generally have a cylindrical shape. In particular, fusion cage 2800 (unlike fusion cage 2900) has surface features 2801 resembling ridges to frictionally engage the surfaces of the adjacent vertebrae. Like the previous embodiment, fusion cages 2800 and 2900 can be generally hollow, having chambers 2805 and 2905, respectively, that extend the lengths of the bodies, in addition to pores 2803 and 2903, respectively, that extend through the surface of the body and can facilitate delivery of an osteogenerative agent. A suitable material for the fusion cages 2800 and 2900 can include a rigid-rod polymer material, which can be strong and resistant to multi-axial forces, including axial loading and torque. In a further embodiment, the fusion cages 2800 and 2900 can include a polymeric material including a rigid-rod polymer. In an example, the polymeric material is a polymer blend including the rigid-rod polymer and a resorbable polymer.

Referring to FIG. 30, an exemplary fusion cage 3000 is illustrated. Fusion cage 3000 can have a generally rectangular contour and can include surface features for frictional engagement on a top surface 3003 and a bottom surface 3005. Moreover, the fusion cage can exhibit a generally hollow center and can include large apertures 3009 on the top surface and the bottom surface, in addition to a threaded aperture 3007 at a proximal end for receiving a threaded fastener to secure the location of the fusion cage within an intervertebral disc space or to assist with inserting the fusion cage 3000 into the intervertebral disc space. A suitable material for the fusion cage 3000 can including a rigid-rod polymer material that can be strong and resistant to multi-axial forces including axial loading and torque. In a further embodiment, the fusion cage 3000 can include a polymeric material including a rigid-rod polymer. In an example, the polymeric material is a polymer blend including the rigid-rod polymer and a resorbable polymer. Referring to FIG. 31, fusion cage 3100 is illustrated as having a generally rectangular structure and a highly porous top surface 3103 and bottom surface 3105.

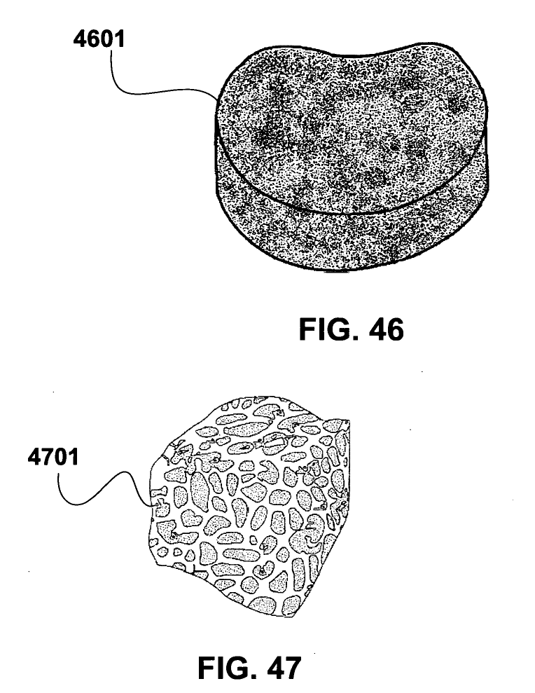

In addition to the fusion cages described in previous embodiments, according to another embodiment, the component can include a porous bone scaffold device, as illustrated in FIGS. 43-47. Generally, porous bone scaffolds can be configured for installation in an intervertebral disc space between two vertebrae, and in particular the porous bone scaffolds provided herein can include a bioactive agent for facilitating bone growth, such as an osteogenerative agent, which can include an osteoconductive or osteoinductive agent. Generally, the porous bone scaffold can include a variety of shapes, generally polygonal, such as cylindrical, rectangular, but not necessarily limited as such, and can include amorphous or kidney-shaped structures. Generally, the porosity of the porous bone scaffolds can be within a range of between about 10-70 vol %, such as 20-50 vol %, or even 20-30 vol %. Additionally, the pore sizes are generally within a range of between about 10-1000 microns, such as within a range of between 250-750 microns.

Referring to FIG. 43, an exemplary porous bone scaffold 4301 is illustrated having a generally rectangular contour. According to an embodiment, the porous bone scaffold can include an outer portion 4003 and an inner portion 4305. In one particular embodiment and as illustrated in FIG. 43, the outer portion 4303 can include a porous structure, and the inner portion 4305 can include an equally or greater porous structure, such as a sponge or reticulated article. Referring to FIG. 44, a porous bone scaffold 4401 can have a generally tetragonal shape and can include a single component reticulated structure. Referring to FIG. 45, a cylindrical-shaped porous bone scaffold 4501 is illustrated having a generally porous outer surface 4503, and a channel 4505 extending through at least a portion of the center of the scaffold structure and in particular extending the entire length of the structure. As will be appreciated, the channel 4505 can be configured to receive a bioactive agent. Referring to FIG. 46, a kidney-shaped porous bone scaffold 4601 is illustrated, having generally a single component reticulated structure. Still, FIG. 47 provides another exemplary embodiment of a porous bone scaffold 4701 which has a generally amorphous form. It will be appreciated that a suitable material for each of the porous bone scaffold structures illustrated in FIG. 43-47 can include a rigid-rod polymer material. In fact, the porous bone scaffold structures can include, in part, or in whole, in one component or multiple components, a rigid-rod polymer material. In particular, a polymeric material including a rigid-rod polymer can be used to form the matrix surrounding the pores. In an example, a removable material or filler can be incorporated with the rigid-rod polymer and dissolved to provide pores. In a particular example, the removable material can be a bioresorbable polymer. In a further example, the removable material can include an agent, such as an osteogenerative agent.

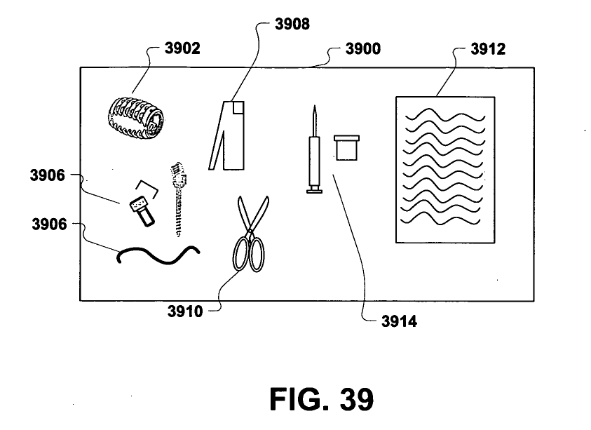

Referring to another component, FIGS. 32-34 illustrate disc prostheses formed to replace a ruptured or degenerated intervertebral disc. Referring to FIG. 32, a disc prosthesis 3201 can be provided that generally has a contour similar to an intervertebral disc. According to an embodiment, the disc prosthesis 3201 can have surface features 3203 for engaging the surface of an adjacent vertebra and holding the prosthetic disc 3201 in place. Additionally, the prosthetic disc 3201 has a core 3205, which can be made of a material that differs in rigidity from that of a surrounding layer 3207. Accordingly, a material such as a rigid-rod polymer material can be suitable for use as the core or the surrounding layer. In a particular embodiment, a rigid-rod polymer material essentially free of filler material is suitable for use as a rigid, load-bearing core. Additionally, in another particular embodiment, a rigid-rod polymer combined with an elastic filler material or polymer blend, is suitable for use as a surrounding layer 3207, where elasticity and wear resistance can be desirable. In particular, the core 3205 can be an elastic polymer and the surrounding layer 3207 can be formed of a polymeric material including a rigid-rod polymer. In another example, the core 3205 can be formed of a polymeric material including a rigid-rod polymer and the surrounding layer 3207 can include an elastomeric polymer. FIG. 33 provides an alternative embodiment of a disc prosthesis 3301. Notably, the disc prosthesis 3301 shown has surface features 3303 that appear to be bumps or mesas extending from the surface of the disc prosthesis 3301 to frictionally engage an adjacent vertebra and hold the disc prosthesis 3301 within the intervertebral disc space. Disc prosthesis 3301 can be a single component disc, which can be made of rigid-rod polymer via a molding method or the like.

FIG. 34 illustrates a further exemplary disc prosthesis 3400. According to the illustrated embodiment, the disc prosthesis can comprise an outer housing 3401 and an inner housing 3403. The inner housing 3401 can be hollow and can generally incorporate a gel or liquid substance suitable for shock-absorption. Alternatively, the inner housing can include a solid substance having elastic properties, such as a polymeric substance. According to a particular embodiment, the outer housing 3401 can include an elastic polymer incorporating a rigid-rod polymer material for additional strength and wear-resistance. The inner housing 3403 can be a more rigid material suitable for engaging adjacent vertebrae and holding the prosthesis in the interspinous disc space. Generally, the inner housing 3403 can incorporate a more rigid material, such as a rigid-rod polymer material. In an example, the inner housing can be formed of a polymeric material including a rigid-rod polymer and having little or no fillers or other additives.

FIG. 35 includes an illustration of an exemplary disc prosthesis 3501. Notably, the disc prosthesis 3501 is a combination of a prosthesis and a fusion cage. The disc prosthesis 3501 can include a rigid material, such as a rigid-rod polymer material suitable for bearing a load of adjacent vertebrae, as well as being highly wear resistant under a constant load. In particular, the disc prosthesis 3501 has a ribbed construction 3503 which can allow the intergrowth of bone. Additionally, the prosthesis 3501 includes a chamber 3505 for housing a carrier of a bioactive agent, such as a sponge or similar device.

Referring to FIG. 36 another disc prosthesis is illustrated according to one embodiment. As illustrated the prosthesis 3601 incorporates a single form solid construction generally having a soft core (not illustrated) housed within a fibrous, wear resistant outer coating. The fibrous coating can be a particularly wear resistant and chemically resistant material to avoid leaching of the inner core material and to maintain the shape of the prosthesis. Accordingly, the inner core can be an elastic material or semi-rigid material, while the outer fibrous coating can be a more rigid material, such as a polymeric material including a rigid-rod polymer.

According to an exemplary embodiment, the implantable device can incorporate a plurality of devices as described in accordance with previous embodiments. Referring to FIG. 37, a disc prosthesis 3701 is illustrated as installed between two vertebrae 3703 and 3705. FIG. 37 also illustrates that in addition to the disc prosthesis 3701, an interspinous process brace 3707 can be installed between an upper process 3709 and a lower process 3711. The disc prosthesis 3701 can be installed and affixed in place between the vertebrae 3703 and 3705 using a retaining plate 3713 that also can include screws 3715 and 3717 to hold the retaining plate in place against and between the vertebrae 3703 and 3705. Particularly, one or more of the components provided in FIG. 37 can incorporate a polymeric material including a rigid-rod polymer.

Referring to FIG. 38, an exemplary embodiment of a disc prosthesis 3801 is illustrated as being fixably engaged between an upper vertebra 3803 and a lower vertebra 3805. The prosthesis 3801 can include a core material that includes a rigid or semi-rigid material such as a rigid-rod polymer material. A cover 3807 can surround the core material and can extend from between the vertebra 3803 and 3505. The cover can be fastened to corresponding vertebra via screws 3809 and 3811. Accordingly, the disc prosthesis can be fixably engage with the vertebrae 3803 and 3805 and fastened to the vertebrae 3803 and 3805. The cover 3807 can be fixably engaged with vertebrae 3803 and 3805. In an example, the cover 3807 can be a material that is sufficiently wear resistance, and as such can incorporate a polymeric material including a rigid-rod polymer.

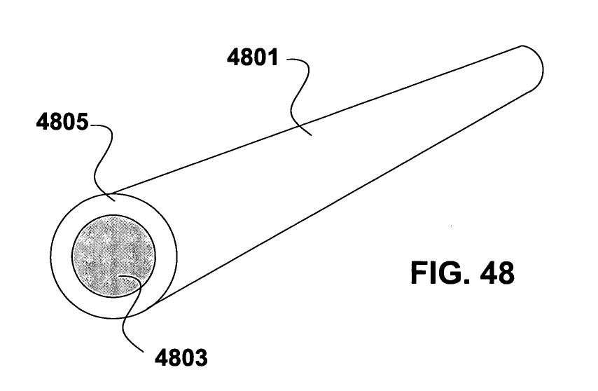

While several embodiment describe above are illustrated as solid components, the components can be formed of multiple layers of material. In particular, the components can include a layer of a metallic, ceramic, or polymeric material and can include a second layer including a polymeric material including a rigid-rod polymer. In a further example, the component can include two layers of different polymeric material including a rigid-rod polymer. In an example illustrated in FIG. 48, an exemplary embodiment of a rod 4801 can include an outer portion 4805 and an inner portion 4803, each respective portion including at least one different material. In one embodiment, the outer portion 4805 and the inner portion 4803 can include various combinations of materials, wherein the material composition of the outer portion 4805 is different than the material composition of the inner portion 4803. In a particular embodiment, the outer portion 4805 can include a metal, metal, alloy or the like and the inner portion 4803 can include a polymer, such as a rigid-rod polymer. In another embodiment, the outer portion 4805 can include a rigid-rod polymer material and the inner portion 4803 can include a metal, metal alloy, or the like. Still, in another particular embodiment, the outer portion 4805 and the inner portion 4803 can include a rigid-rod polymer material such that the composition of the respective portions can include a different filler material.

### Treatment Kit

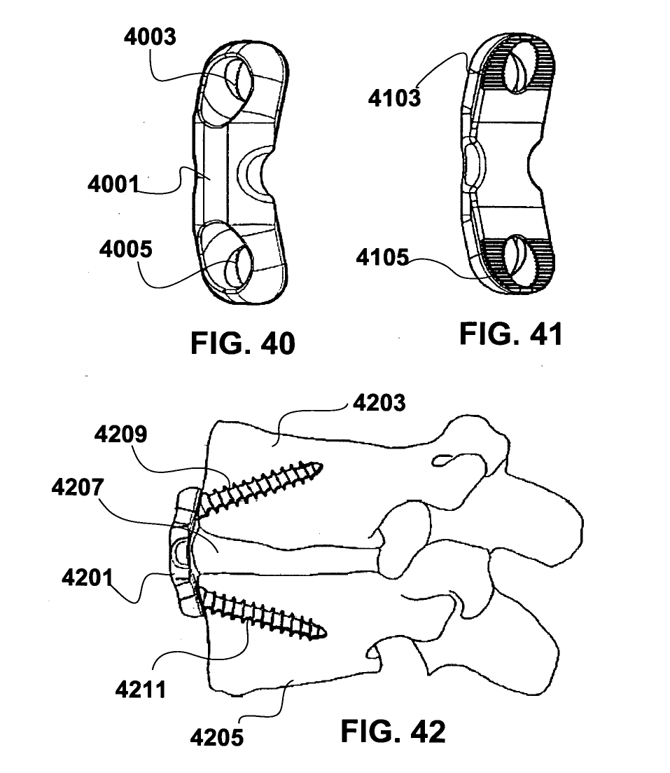

An implantable device described herein or components thereof can be included in a kit. In an exemplary embodiment, FIG. 39 includes an illustration of an exemplary kit 3900. For example, the kit 3900 can include a device component 3902. The device component 3902 can be adapted to engage a portion of the spine, such as a vertebra. In a particular example, the component 3902 can include a fastener, a rod, a fusion cage, prosthetic disc, or any of the above described embodiments. In addition or alternatively, the kit 3900 can include a strand material 3904 adapted to engage a joint, such as a zygapophysial joint, or a process, such as a spinous process or an articular process.

In addition, the kit 3900 can include a tool to further adapt the component 3902 or the strand material 3904, such as scissors 3910 or a cutting tool. For example the component 3902 or the strand material 3904 can be adapted based on the location or the size of the processes it is to engage.

In another example, the kit 3900 can include one or more fasteners 3906. For example, the kit 3900 can include staples, screws, or crimp fasteners to secure the component 3902 or the strand material 3904. In a further example, the kit 3900 can include a tool 3908 to secure the component 3902 or the strand material 3904. For example, the tool 3908 can be a stapler or a screwdriver to secure the component 3902 to a process or a vertebral body. In another example, the tool 3908 can include a crimp tool to secure the strand material 3904 or the component 3902 to itself.

In an additional example, the kit 3900 can include an agent 3914. For example, the kit 3900 can include an agent 3914 and a syringe for injecting the agent 3914 into the component 3902, or a portion of the spine. In another example, the syringe can include a gel that includes the agent 3914 for injection into a space proximate to the component 3902 and a portion of the spine. In an alternative embodiment, the syringe can include an adhesive, gel material, or bone cement to facilitate fusion of the component 3902 and a vertebra.

In a particular embodiment, the kit 3900 includes an indication of the use of the component 3902 or the strand material 3904. For example, an indicator 3912 can identify the kit 3900 as a repair or support system for a portion of the spine. In another example, the indicator 3912 can include contraindications for use of the kit 3900 and materials 3902 and 3904. In a further example, the indicator 3912 can include instructions, such as instructions regarding the installation of the device and materials 3902 and 3904.

In an exemplary embodiment, the kit components can be disposed in a closed container, which can be adequate to maintain the contents of the container therein during routine handling or transport, such as to a healthcare facility or the like.

### Method of Implanting

The implantable devices described herein can be generally implanted subcutaneously in proximity to or within the spine. For example, the implantable device can be implanted within an intervertebral space, within or across a zygapophysial joint, between spinous processes, or across the outer surface of two vertebra. To implant the device, a surgeon can approach the spine from one of several directions including posteriorally, through the abdomen, or laterally.

Generally, the implantable device includes at least one component. When the implantable device includes more than one component, the implantable device can be prepared by assembling the device. Alternatively, the device can be assembled as parts are engaged with the spine. In another example, the implantable device can be prepared by applying an agent to the device or impregnating the device with an agent. In a further example, the implantable device can be prepared by configuring the device, such as adjusting the size of the device.

For particular devices, the space between two vertebrae can be extended to permit insertion of the device. Alternatively, the device can be implanted and the implanted device can be extended to provide the desired spacing between vertebrae.

Once the device is implanted, a surgeon can remove tools used in the insertion process and close the surgical wound.

### CONCLUSION

With embodiments of the devices described above, the condition of a spine, and in particular, a set of discs and zygapophysial joints, can be maintained, repaired, or secured. Such a device can be used to limit further deterioration of a degrading zygapophysial joint or intervertebral disc. In another example, such a device can be used to secure the zygapophysial joint or the intervertebral disc during fusion of the associated articular processes or vertebral bodies. In an additional example, the device can be used to permit healing of capsular ligaments, the zygapophysial joint, or the intervertebral disc after an acute stress injury.

In a particular embodiment, the device can act to limit undesired movement of the processes and the associated vertebra relative to each other. As such, the device can reduce the likelihood of further injury to soft tissue associated with the spine, reduce pain associated with spine damage, and complement other devices, such as implants and fusion devices.

Particular embodiments of the implantable device including a component formed of a polymeric material including a rigid-rod polymer can advantageously provide improved device performance. For example, a fusion device including a polymeric material including a rigid-rod polymer matrix can provide osteoconductive surfaces while also providing a strong structural support. Particular rod devices and securing devices can advantageously be scored to break without undesirable elongation, maintaining thread integrity, and without undesirable back-lash.

Particular embodiments of an implantable device can be advantageously formed of a polymeric material including a rigid-rod polymer to prevent stress shielding. Particular rigid-rod polymer materials can provide suitable strength while having suitable modulus, in contrast to traditional polymer, metallic, or ceramic materials. In particular, a polymeric material formed essentially of a rigid-rod polymer can provide desirable properties.

Moreover, particular species of rigid-rod polymer provide a combination of advantageous properties to polymeric materials forming spinal implant devices. In an exemplary embodiment, the rigid-rod polymer can be a thermoplastic rigid-rod polymer. In addition, particular rigid-rod polymers provide substantially isotropic mechanical properties. In particular, a polymeric material including a thermoplastic isotropic rigid-rod polymer, and particularly an amorphous thermoplastic isotropic rigid-rod polymer, can advantageously be used in components of an implantable device, alone or as a polymer matrix.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

1. An implantable device comprising:
a first component configured to fixably attach to a first vertebra to secure the first vertebra in a position relative to a second vertebra, the component comprising a polymeric material including a rigid-rod polymer.

2. The implantable device of claim 1, wherein the first component is configured to fixably attach to a second vertebra.

3. The implantable device of claim 1, wherein the first component is configured to be located at least partially in an intervertebral space between the first vertebra and the second vertebra.

4. The implantable device of claim 1, wherein the first component is configured to be located at least partially in a facet space between the first vertebra and the second vertebra.

5. (canceled)

6. (canceled)

7. (canceled)

8. (canceled)

9. The implantable device of claim 1, wherein the first component comprises an engagement surface having surface features to frictionally engage the first vertebrae.

10. (canceled)

11. The implantable device of claim 1, wherein the first component is porous.

12. The implantable device of claim 1, wherein the first component is hollow.

13. The implantable device of claim 1, wherein the first component comprises at least one surface having a coating comprising a bioactive agent.

14. The implantable device of claim 13, wherein the bioactive agent comprises an osteogenerative agent.

15. (canceled)

16. (canceled)

17. (canceled)

18. (canceled)

19. (canceled)

20. The implantable device of claim 1, wherein the polymeric material consists essentially of the rigid-rod polymer.

21. The implantable device of claim 1, wherein rigid-rod polymer forms a matrix and wherein the polymeric material further includes a filler material comprising a ceramic, a metal, a polymer, or any combination thereof.

22. (canceled)

23. (canceled)

24. (canceled)

25. (canceled)

26. (canceled)

27. (canceled)

28. The implantable device of claim 21, wherein the polymeric material includes a polymer blend including the rigid-rod polymer and at least one other polymer.

29. (canceled)

30. (canceled)

31. (canceled)

32. (canceled)

33. (canceled)

34. (canceled)

35. (canceled)

36. The implantable device of claim 1, wherein the rigid-rod polymer comprises a phenylene-based homopolymer or copolymer.

37. The implantable device of claim 36, wherein the rigid-rod polymer includes poly(phenylene benzobisthiazole), poly(phenylene benzobisoxazole), poly(phenylene benzimidazole), poly(phenylene terephthalate), poly(benzimidazole), or any combination thereof.

38. (canceled)

39. The implantable device of claim 1, wherein the polymeric material has an average tensile modulus at room temperature (23° C.) of not less than about 5.00 GPa.

40. (canceled)

41. (canceled)

42. (canceled)

43. The implantable device of claim 1, wherein the polymeric material has a specific gravity at room temperature of less than about 1.40.

44. (canceled)

45. (canceled)

46. (canceled)

47. An implantable device comprising:
a component configured to fixably attach to a first vertebra, the component comprising a polymer material having a specific gravity of not greater than about 1.40 and an ultimate tensile strength at room temperature (23° C.) of at least about 125 MPa.

48. The implantable device of claim 47, wherein the polymer material is homogeneous.

49. The implantable device of claim 47, wherein the polymer material consists essentially of a rigid-rod polymer.

50. The implantable device of claim 47, wherein the polymer material has a specific gravity of less than about 1.30.

51. (canceled)

52. The implantable device of claim 47, wherein the polymer material has an ultimate tensile strength at room temperature (23° C.) of at least about 150 MPa.

53. The implantable device of claim 52, wherein the polymer material has an ultimate tensile strength at room temperature (23° C.) of at least about 200 MPa

54. The implantable device of claim 47, wherein the polymer material has an average tensile modulus at room temperature (23° C.) of at least about 5.00 GPa.

55. (canceled)

56. The implantable device of claim 47, wherein the polymer material has an average flexural yield strength at room temperature (23° C.) of at least about 220 MPa.

57. (canceled)

58. The implantable device of claim 47, wherein the polymer material has an average flexural modulus at room temperature (23° C.) of at least about 5.00 GPa.

59. (canceled)

60. The implantable device of claim 47, wherein the polymer material has an average compressive yield strength at room temperature (23° C.) of at least about 230 MPa.

61. (canceled)

62. (canceled)

63. The implantable device of claim 47, wherein the polymer material has substantially isotropic mechanical properties.

64. The implantable device of claim 47, wherein the polymer material has a glass transition temperature of at least about 145° C.

65. (canceled)

66. (canceled)

67. (canceled)

68. (canceled)

69. (canceled)

70. (canceled)

71. (canceled)

72. (canceled)

73. A medical kit comprising:
a component of an implantable device, the component configured to fixably attach to a first vertebra to secure the first vertebra in a position relative to a second vertebra, the component comprising a polymeric material including a rigid-rod polymer; and
a tool configured for use in association with fixably attaching the component to the first vertebra.

74. (canceled)

75. (canceled)

76. (canceled)

77. (canceled)

78. (canceled)

79. (canceled)

80. (canceled)

81. (canceled)
